(19) United States
(12) Patent Application Publication
(10) Pub. No.: US 2015/0252375 A1
(43) Pub. Date: Sep. 10, 2015

(54) TRANSCRIPTIONAL GENE SILENCING OF ENDOGENES IN PLANTS

(71) Applicant: THE ROCKEFELLER UNIVERSITY, New York, NY (US)

(72) Inventors: Nam-Hai Chua, New York, NY (US); Shulin Deng, New York, NY (US); Hongzheng Dai, Ellicott City, MD (US); Qi-Wen Niu, Staten Island, NY (US); Huan Wang, New York, NY (US); Catalina Arenas Huertero, Merced (MX)

(73) Assignee: THE ROCKEFELLER UNIVERSITY, New York, NY (US)

(21) Appl. No.: 14/426,096

(22) PCT Filed: Aug. 27, 2013
(86) PCT No.: PCT/US2013/056865

§ 371 Date: Mar. 4, 2015

Related U.S. Application Data
(60) Provisional application No. 61/698,203, filed on Sep. 7, 2012.

(51) Int. Cl. C12N 15/82

(52) U.S. Cl. CPC C12N 15/8218; C12N 15/8261

(57) ABSTRACT

The present invention relates to transcriptional gene silencing (TGS) of endogenes in plants, plant tissue and plant cells. More specifically, the present invention relates to nucleic acid constructs that are capable of TGS of endogenes in plants, plant tissue and plant cells. The present invention further relates to methods of reducing endogenous gene expression in plants, plant tissues or plant cells by TGS using the nucleic acid constructs of the invention.

### CROSS-REFERENCE TO RELATED APPLICATIONS

The present application is related to and claims priority to U.S. Provisional Patent Application Ser. No. 61/698,203, filed 7 Sep. 2012. This application is incorporated herein by reference.

### SEQUENCE SUBMISSION

The present application is being filed along with a Sequence Listing in electronic format. The Sequence Listing is entitled 2312130PCTSequenceListing.txt, created on 26 Aug. 2013 and is 14 kb in size. The information in the electronic format of the Sequence Listing is incorporated herein by reference in its entirety.

### BACKGROUND OF THE INVENTION

The present invention relates to transcriptional gene silencing (TGS) of endogenes in plants, plant tissue and plant cells. More specifically, the present invention relates to nucleic acid constructs that are capable of TGS of endogenes in plants, plant tissue and plant cells. The present invention further relates to methods of reducing endogenous gene expression in plants, plant tissues or plant cells by TGS using the nucleic acid constructs of the invention.

The publications and other materials used herein to illuminate the background of the invention or provide additional details respecting the practice, are incorporated by reference, and for convenience are respectively grouped in the Bibliography.

More than a decade ago, Matzke and colleagues reported the relationship between transcriptional gene silencing (TGS) of a transgene promoter (NOS) in plants and methylation on the promoter DNA (Matzke et al., 1989). Since then, mechanisms underlying TGS in plants have been extensively studied for both transgenes and endogenes in monocots and dicots (for reviews, see (Matzke and Matzke, 2004; Eamens et al., 2008; Matzke et al., 2009)). The current view of TGS suggests that 21-24 nt small RNAs (sRNAs) generated by double-stranded RNA processing machinery (AGO4, DCL3) are targeted to genomic regions with sequence homology. These sRNAs guide DNA methylation by the action of DNA methytransferases (DRM1/2, MET1, CMT3) and presumably this is followed by histone modifications involving histone methytransferase and deacetylase so as to establish a repressive chromatin state at the methylated locus.

Early studies using transgenes as a model system uncovered some basic features related to TGS in plants (Mette et al., 2000; Jones et al., 2001; Sijen et al., 2001). These studies demonstrated that long hairpin structures are required to generate sRNAs targeted to transgene promoters. DNA analysis of the silenced transgenes showed increases in both symmetrical and asymmetrical cytosine methylations and the involvement of MET1 in the maintenance of the silenced locus. Similarly, long inverted repeats (IR) have also been shown to mediate efficient TGS of a 35S promoter in rice (Okano et al., 2008). Notably, single-stranded silencers in the sense (S) or antisense (AS) orientation were inefficient in producing sRNAs and generating TGS in tobacco and Arabidopsis (Mette et al., 2000).

Although the sequence of events surrounding TGS of transgenes is relatively well defined it is not known whether similar steps also apply to TGS of endogenous loci (endogenes hereinafter). Surprisingly, only a few cases of TGS of endogenous loci have been reported to-date, all of which used IR RNA's Cigan et al. (2005) have reported successful and strong silencing of two maize endogenous genes (Mark Cigan et al., 2005). Moderate silencing was seen for a few endogenes in petunia, potato and rice (Sijen et al., 2001; Heilersig et al., 2006; Okano et al., 2008). The inefficiency of IR RNA's to trigger TGS of endogens was well illustrated in a comprehensive study using the model monocot rice. Interestingly, 6 out of 7 endogenes examined in rice appeared to be recalcitrant to silencing by sRNAs generated from IR RNA structures (Okano et al., 2008). By contrast, in the same experiment it was shown that a 35S promoter could be efficiently silenced by IR RNA targeted to this promoter. Other unsuccessful trials (phytoene desaturase, (PDS) and Chalcone synthase (CHS)) have also been reported in Arabidopsis (Eamens et al., 2008). Together, these results suggest endogenous loci may possess some intrinsic properties that can prevent unexpected TGS; alternatively, there is the need to explore more efficient silencing strategies for endogenous loci. Other than the observed low success rate of gene silencing at endogenous loci, there is also a discrepancy between TGS and DNA methylation as well as between DNA methylation and histone modifications for endogenes (Okano et al., 2008). For most of the cases in rice, IR RNA's targeted to promoter regions could trigger DNA methylation of homologous sequences but they failed to induce chromatin modifications and TGS (Okano et al., 2008).

It is desired to develop nucleic acid constructs and methods of transcriptional gene silencing in plants.

### SUMMARY OF THE INVENTION

The present invention relates to transcriptional gene silencing (TGS) of endogenes in plants, plant tissue and plant cells. More specifically, the present invention relates to nucleic acid constructs that are capable of TGS of endogenes in plants, plant tissue and plant cells. The present invention further relates to methods of reducing endogenous gene expression in plants, plant tissues or plant cells by TGS using the nucleic acid constructs of the invention.

In a first aspect, the present invention provides a nucleic acid construct comprising a plant operable promoter as described herein operably linked to a nucleic acid silencer molecule described herein. The nucleic acid construct may optionally include other regulatory sequences, such as 3′ regulatory sequences, or other sequences as described herein. The nucleic acid silencer molecule of the present invention comprises a promoter region of a plant endogene target, i.e., a plant endogene to be downregulated via TGS. The nucleic acid silencer molecule encodes either a single-stranded silencer which is an RNA molecule transcribed from the nucleic acid construct or an inverted repeat silencer transcribed from the nucleic acid construct. The single-stranded silencer or inverted repeat silencer provides TGS of endogenes in plants, plant tissues and plant cells. In one embodiment, the single-stranded silencer is an RNA molecule that is produced from a promoter region of a plant endogene target (i.e., a nucleic acid silencer molecule) that is in an antisense orientation with respect to the plant operable promoter in the nucleic acid construct. In another embodiment, the single-stranded silencer is an RNA molecule that is produced from a promoter region of a plant endogene target (i.e., a nucleic acid silencer molecule) that is in a sense orientation with respect to the plant operable promoter in the nucleic acid construct. In each of these embodiments, the construct, nucleic acid silencer molecule and single-stranded silencers are in the absence of inverted repeat structures, i.e., no inverted repeat structures or inverted repeats are present in the nucleic acid construct and products produced from it. In yet another embodiment, the nucleic acid silencer is an RNA molecule that is produced from a promoter region of a plant endogene target that is provided in duplicate and arranged in an inverted repeat configuration with respect to the plant operable promoter in the nucleic acid construct. Expression of the nucleic acid silencer molecule produces an initial single stranded RNA. This single stranded RNA may be converted to a double stranded RNA by cellular mechanisms or as a result of the inverted repeat structure.

In one embodiment, the nucleic acid silencer molecule comprises nucleotides upstream of the transcription start site of the target endogene. In another embodiment, the nucleic acid silencer molecule comprises nucleotides upstream of the transcription start site and nucleotides downstream of the transcription start site of the target endogene. In some embodiments, the nucleic acid silencer molecule comprises a promoter region of about 300 contiguous nucleotides to about 1500 contiguous nucleotides of the endogene. In other embodiments, the nucleic acid silencer molecule comprises a promoter region of about 400 contiguous nucleotides to about contiguous 1200 nucleotides of the endogene. In additional embodiments, the nucleic acid silencer molecule comprises a promoter region of about 425 contiguous nucleotides to about 1100 contiguous nucleotides of the endogene. In further embodiments, the nucleic acid silencer molecule comprises a promoter region of about 425 contiguous nucleotides to about 1075 contiguous nucleotides of the endogene.

Any promoter that is operable in a plant may be used in the nucleic acid construct to drive expression of the nucleic acid silencer molecule. In some embodiments, the promoter is a single copy of a plant operable promoter, including those described herein. In other embodiments, the promoter is a double copy of a plant operable promoter to make a homologous double promoter. In further embodiments, the promoter is a combination of two different promoters to make a heterologous double promoter.

In a second aspect, the present invention provides a transgenic plant cell comprising the nucleic acid construct. In one embodiment, the nucleic acid construct is stably integrated into the genome of the transgenic plant cell. In another embodiment, the nucleic acid is expressed in the transgenic plant cell.

In a third aspect, the present invention provides a transgenic plant comprising the nucleic acid construct. In one embodiment, the nucleic acid construct is stably integrated into the genome of the transgenic plant. In another embodiment, the nucleic acid is expressed in the transgenic plant.

In a fourth aspect, the present invention provides a method of reducing endogenous gene expression in plants, plant tissues or plant cells via transcriptional gene silencing. In one embodiment, the method comprises transfecting a plant cell with the nucleic acid construct to produce a transgenic plant cell as described herein. The method further comprises expressing the nucleic acid in the transgenic plant cell as described herein. The expressed nucleic acid, i.e., an RNA single stranded sense silencer, an RNA single stranded antisense silencer or an IR silencer described herein, is cleaved in the transgenic plant cell to produce one or more small RNAs (sRNAs) that induces transcriptional gene silencing to reduce expression of the gene of interest. In some embodiments, expression of the nucleic acid silencer molecule produces an initial single stranded RNA. This single stranded RNA may be converted to a double stranded RNA before processing to produce sRNAs by cellular mechanisms or as a result of the inverted repeat structure. The method may optionally include preparing a nucleic acid construct encoding a single stranded silencer or an IR silencer as described herein. In another embodiment, the method comprises regenerating a transgenic plant from the transgenic plant cell. In this embodiment, the nucleic acid is expressed in the transgenic plant. The expressed nucleic acid is cleaved in the transgenic plant cell to produce one or more sRNAs that induces transcriptional gene silencing to reduce expression of the gene of interest.

In a fifth aspect, the present invention provides nucleic acid constructs and methods to identify and obtain other TGS silencers from promoter regions of endogenes. According to this aspect, the nucleic acid construct is one that is suitable for transformation of a plant species for which it is desired to identify a TGS silencer. In one embodiment, the nucleic acid construct may be included in a vector. In some embodiments, the vector is suitable for Agrobacterium-mediated transformation. In other embodiments, the vector may be suitable for biolistic-mediated transformation. Other suitable vectors for plant transformations are well known to the skilled artisan. In another embodiment, the nucleic acid construct may be used directly for the transformation of a plant according to techniques well known to the skilled artisan. The nucleic acid construct comprises a plant operable promoter operatively linked to a putative nucleic acid silencer molecule operatively linked to plant operable 3′ regulatory region. In one embodiment, the putative nucleic acid silencer molecule comprises a promoter region of a plant endogene target which is to be tested for transcriptional gene silencing. In some embodiments, the putative nucleic acid silencer molecule is in a sense orientation with respect to the plant operable promoter as described herein. In other embodiments the putative nucleic acid silencer molecule is in an antisense orientation with the respect to the plant operable promoter. In further embodiments, the putative nucleic acid silencer molecule contains an inverted repeat or inverted repeat structure as described herein. In one embodiment, the plant operable promoter is a double promoter, such as a double 35S CMV promoter. In an additional embodiment, the plant operable promoter is a single promoter, such as a single 35S CMV promoter. In one embodiment, the 3′ regulatory sequence is a TRV2 3′ sequence. In yet another embodiment, the 3′ regulatory region is a polyA addition sequence. In one embodiment, the polyA addition sequence is a NOS polyA. Further according to this aspect, suitable TGS silencers are identified by a method which comprises the steps of preparing a nucleic acid construct comprising a putative nucleic acid silencer molecule of an endogene of interest as described herein, transforming a cell or tissue of a plant species of interest with the nucleic acid construct and determining whether the putative nucleic acid silencer molecule is processed by the transformed cell or tissue of the plant species to produce silencing of the endogene of interest. If the endogene is silenced, then the putative nucleic acid silencer molecule of the endogene of interest is identified as a TGS silencer. In one embodiment, the determination is made by culturing the transformed plant cell for expression of the putative nucleic acid silencer molecule and testing for transcriptional gene silencing in the cultured transformed plant cell or tissue. In another embodiment, the determination is made by regenerating a transformed plant from the transformed plant cell or tissue and testing for transcriptional gene silencing in the transformed plant. The regeneration of transformed plants is performed according to techniques well known to the skilled artisan.

### BRIEF DESCRIPTION OF THE FIGURES

FIGS. 1a-1d show that a single strand silencer and an IR silencer can induce transcriptional silencing at the TMM locus. FIG. 1a: Shows a schematic representation of the silencer constructs. A 475 bp (−483 to −9) fragment of the TMM promoter region was used as a silencer. a1: Silencers without polyA addition sequence. The TMM transcriptional start site (TSS)) of the TMMpromoter region was used as silencer, which was driven by double 35S either in sense (S, sense silencer), or antisense (AS, antisense silencer) direction. Inverted repeat silencer (IR) was also made as a control. a2: Silencers with NOS polyA addition sequence. Silencers (S, AS and IR) were transcribed from a 35S promoter. All 3′ ends of constructs carried a NOS polyA addition sequence. FIG. 1b: Shows typical clustering stomata morphology observed in sense silencer line (S21), antisense silencer line (AS10), and inverted repeat silencer line (IR8). WT, wild type Col-0. Bar=10 μm. FIG. 1c and FIG. 1d: Show reduced TMM transcripts level by pTMM silencers. FIG. 1c: Shows reduced transcripts level of TMM in T1 transformed plants. For each silencer construct, 50-100 T1 transformed plants were pooled and analyzed. FIG. 1d: Shows TMM mRNA reduction in representative S silencer lines (S21, S26), AS silencer lines (AS4, AS10), and IR silencer lines (IR8, IR44). AS10NI and IR8NI indicate progeny plant lines which did not carry any transgene from a T2 segregation population of AS10 and IR8, respectively. Vect.: vector control. Data shown are mean of three technical repeats±SD (standard deviation). Similar results were obtained in another independent experiment.

FIG. 2 shows T-DNA insertion locations of single-stranded silencers in transgenic plants. In each line, the T-DNA insertion position is indicated by an arrow. Schematic diagram shows disrupted Arabidopsis genes. White box, UTR; black box, exon region; black line, intron region. At5G54045, a pseudogene, is represented in gray. The dotted line between At5G09730 and At5G54045 indicates a very large genomic distance.

FIGS. 3a and 3b show clone number and size distribution of sRNAs mapped to the TMM promoter region. FIG. 3a: Normalized clone number of sRNAs mapped to the TMM promoter. The TMM promoter-related sRNAs were normalized to the total number of 21-24nt sRNAs that can be mapped to Arabidopsis genome. Plus and Minus refer to sense strand and antisense strand, respectively, rpm means read per million reads. FIG. 3b: Size distribution of sRNAs mapped to the TMM promoter.

FIGS. 4a-4e show the DNA methylation profiles at the endogenous TMM promoter and transgenic silencer region. FIG. 4a: Shows the bisulfate sequencing region of targeted endogenous TMM promoter. FIG. 4b, FIG. 4c and FIG. 4d: Show DNA methylation levels at CG, CHG (H=A, C, or T), and CHH type of Cytosine, respectively. FIG. 4e: Shows DNA methylation at the transgenic silencer region. AS2, a line carrying the transgenic silencer without TGS, was used as a control.

FIGS. 5a and 5b show that the DNA methylation at the TMM promoter region is silencer dependent. Endonuclease McrBc cleaves DNA containing (G/A)^{m}C(N₄₀₋₃₀₀₀) (G/A)^{m}C. FIG. 5a: Shows the 5′ and 3′ region of the TMM promoter that were tested for DNA methylation. FIG. 5b: qPCR results with or without McrBc digestion. WT, wild type Col-0; AS4, AS10, antisense silencer lines. AS10NI, plants are derived from heterozygous AS10 and contain no silencer. U, without McrBc treatment. D, with McrBc treatment.

FIGS. 6a-6d show histone H3 modification patterns at the TMM promoter and coding region in plants silenced by AS or IR silencers. FIG. 6a: Q-PCR primers designed to amplify DNA fragments (around 100 bp) corresponding to three different regions of the TMM promoter and coding sequences as indicated. FIG. 6b: Histone H3 modification patterns at the TMM promoter 5′ region. FIG. 6c: Histone H3 modification patterns at the TMM promoter 3′ region. FIG. 6d: Histone H3 modification patterns at the TMM coding region. Ace: H3K9/K14 acetylation. K4me3: Histone H3 Lys 4 trimethylated (H3K4) form. K9me3: Histone H3 Lys 9 (H3K9) trimethylated form. K27me3: Histone H3 Lys 27 (H3K27) trimethylated form.

FIGS. 7a-7d show that single strand FHY1 silencer targeting on FHY1 promoter can induce the fhy1-phenotype. FIG. 7a: Longer hypocotyl length of S silencer lines (S3, S4) and antisense silencer lines (AS8, AS11) compared to wild type (WT, Col-0) under 5 μmol/m²/s far red light (FR) treatment for four days. fhy1-3, FHY1 loss of function mutant. Bar=5 mm. FIG. 7b: Is the quantification of FIG. 7a, data shown as mean hypocotyl lengths of 20 individuals±SD (standard deviation). FIG. 7c: Relative FHY1 messenger RNA transcript levels detected by q-RT-PCR using gene-specific primers. FIG. 7d: FHY1 DNA methylation. Histograms are the average of triplicate assays and the bars indicate SD.

FIGS. 8a-8d show the classification of the tmm-mutant phenotype. FIG. 8a: WT, Col-0. FIG. 8b: Weak phenotype. FIG. 8c: Intermediate phenotype. FIG. 8d: Strong phenotype.

FIGS. 9a-9c shows the sRNA's size distribution. The number on the X-axis refers to the size of the sRNA's (nt). (a), (b), (c), (d), (e), (f), (g) and (h) represent WT, S21, S26, AS4, AS10, AS10NI, IR8 and IR8NI, respectively.

FIG. 10 shows the Southern blot analysis of sRNAs. (a), ethidium bromide (EtBr)-stained rRNA was used as a loading control. (b), minus strand derived sRNA detected by sense probe. (c), plus strand derived sRNA detected by the antisense probe.

FIGS. 11a-11f show the DNA methylation pattern at the endogenous TMM promoter region. FIG. 10a, FIG. 10b and FIG. 10c show CG, CHG, and CHH methylation patterns of sense silencer transgenic lines. FIG. 10d, FIG. 10e and FIG. 10f show CG, CHG, and CHH methylation patterns of antisense silencer and inverted repeated silencer transgenic lines. The numbers on the X-axis refer to the nucleotide positions upstream of the TMM transcription start site.

FIGS. 12a-12c show DNA methylation patterns on the transgenic silencer region of AS silencer transgenic plants. (A), (b) and (c) show CG (FIG. 11a), CHG (FIG. 11b) and CHH (FIG. 11c) methylation patterns, respectively. The numbers on the X-axis refer to the nucleotide positions downstream of the 35S promoter transcription start site.

FIGS. 13a-13b show that single strand silencer can induce hfr1-phenotype as well as inverted repeat silencer targeting on HFR1 promoter. FIG. 13a: Hypocotyl length measured after 1.5 μmol/m²/s far red light treatment for 4 days. FIG. 13b: HFR1 transcripts level reduction detected by qPCR. WT, wild type Col-0. S2, S8, transgenic lines carrying sense silencer. AS2, AS5, AS6, transgenic lines carrying antisense silencer. IR2, IRS, IR6 transgenic lines carrying inverted repeat silencer. HFR1 loss of function mutant hfr1 used here was hfr1-201.

FIGS. 14a and 14b show DNA methylation patterns at the endogenous FHY1 promoter region. FIG. 14a, FIG. 14b and FIG. 14c show CG, CHG, and CHH methylation patterns, respectively. Position refers to FHY1 transcription star site.

FIGS. 15a and 15b show that single strand PhyB silencer targeting on PhyB promoter can induce phyB-phenotype. FIG. 15a: Longer hypocotyl length of S silencer lines (S11, S12, S13) and antisense silencer lines (AS9, AS10, AS11) than wild type (WT, Col-0) under 15 μmol/m²s red light (RL) treatment for 5 days. phyB-9, PhyB loss of function mutant. FIG. 15b: Is the quantification of FIG. 15a, data shown as mean of 20 individuals±SD (standard deviation).

### DETAILED DESCRIPTION OF THE INVENTION

The present invention relates to transcriptional gene silencing (TGS) of endogenes in plants, plant tissue and plant cells. More specifically, the present invention relates to nucleic acid constructs that are capable of TGS of endogenes in plants, plant tissue and plant cells. The present invention further relates to methods of reducing endogenous gene expression in plants, plant tissues or plant cells by TGS using the nucleic acid constructs of the invention.

Unless defined otherwise, all technical and scientific terms used herein have the same meaning as is commonly understood by one of skill in the art to which the invention belongs.

The terms “polynucleotide,” “nucleotide sequence,” and “nucleic acid” are used to refer to a polymer of nucleotides (A, C, T, U, G, etc. or naturally occurring or artificial nucleotide analogues), e.g., DNA or RNA, or a representation thereof, e.g., a character string, etc., depending on the relevant context. A given polynucleotide or complementary polynucleotide can be determined from any specified nucleotide sequence.

A polynucleotide, polypeptide or other component is “isolated” when it is partially or completely separated from components with which it is normally associated (other proteins, nucleic acids, cells, synthetic reagents, etc.). A nucleic acid or polypeptide is “recombinant” when it is artificial or engineered, or derived from an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

The term “nucleic acid construct” or “polynucleotide construct” means a nucleic acid molecule, either single- or double-stranded, which is isolated from a naturally occurring gene or which has been modified to contain segments of nucleic acids in a manner that would not otherwise exist in nature. The term nucleic acid construct is synonymous with the term “expression cassette” when the nucleic acid construct contains the control sequences required for expression of a sequence of the present invention.

The term “control sequences” is defined herein to include all components, which are necessary or advantageous for the expression of a polynucleotide of the present invention. Each control sequence may be native or foreign to the polynucleotide sequence. At a minimum, the control sequences include a promoter and transcriptional stop signals. The control sequences may be provided with linkers for the purpose of introducing specific restriction sites facilitating ligation of the control sequences to the nucleotide sequence.

The term “operably linked” is defined herein as a configuration in which a control sequence is appropriately placed at a position relative to the nucleotide sequence of the nucleic acid construct such that the control sequence directs the expression of a polynucleotide of the present invention.

In the present context, the term “expression” includes transcription of the polynucleotide. In the present context, the term “expression vector” covers a DNA molecule, linear or circular, that comprises a polynucleotide of the invention, and which is operably linked to additional segments that provide for its transcription.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

The term “heterologous” as used herein describes a relationship between two or more elements which indicates that the elements are not normally found in proximity to one another in nature. Thus, for example, a polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). An example of a heterologous polypeptide is a polypeptide expressed from a recombinant polynucleotide in a transgenic organism. Heterologous polynucleotides and polypeptides are forms of recombinant molecules.

The term “transfecting” as used herein refers to the deliberate introduction to a nucleic acid into a cell. Transfection includes any method known to the skilled artisan for introducing a nucleic acid into a cell, including, but not limited to, Agrobacterium infection, ballistics, electroporation, microinjection and the like.

The term “nucleic acid silencer molecule” as used herein refers to a part of a nucleic acid construct in accordance with the present invention that comprises a promoter region of a target plant endogene. The nucleic acid silencer molecule is transcribed to initially produce a single-stranded RNA that is processed in the plant cell to produce small RNAs (sRNAs) that induce transcriptional gene silencing of the target plant endogene. The nucleic acid silencer molecule may be placed in a sense orientation or in an antisense orientation with respect to the plant operable promoter of the nucleic acid construct or it may be placed in an inverted repeat structure with respect to the plant operable promoter of the nucleic acid construct.

The term “single-stranded sense silencer” or “single-stranded S silencer” as used herein refers to a single stranded RNA produced by a nucleic acid silencer molecule in the sense orientation with respect to the promoter.

The term “single-stranded antisense silencer” or “single-stranded AS silencer” as used used herein refers to a single stranded RNA produced by a nucleic acid silencer molecule in the antisense orientation with respect to the promoter.

The term “inverted repeat silencer” or “IR silencer” as used herein refers to a RNA molecule produce by a nucleic acid silencer molecule having a two copies of the target endogene promoter sequence, one copy inverted with respect to the second copy and preferably separated by a spacer.

“Reduced gene expression” means that the expression of a plant endogene is reduced in a transgenic plant cell or transgenic plant containing a nucleic acid silencer molecule stably integrated in its genome when compared to a plant cell or plant which does not contain the nucleic acid silencer molecule. “Reduced gene expression” may involve a reduction of expression of a plant endogene by at least 10%, 20%, 30%, 40%, 50%, 60%, 70%, 75%, 80%, 85%, 90%, 95% or 100%.

Thus, in a first aspect, the present invention provides a nucleic acid construct comprising a plant operable promoter, as described herein operably linked to a nucleic acid silencer molecule described herein. The nucleic acid construct may optionally include other regulatory sequences, such as 3′ regulatory sequences, or other sequences as described herein. The nucleic acid silencer molecule of the present invention comprises a promoter region of a plant endogene target, i.e., a plant endogene to be downregulated via TGS. The nucleic acid silencer molecule encodes either a single-stranded silencer or an inverted repeat (IR) silencer, either of which is initially an RNA molecule that is transcribed from the nucleic acid construct. The single-stranded silencer and IR silencer provide TGS of endogenes in plants, plant tissues and plant cells. In one embodiment, the single-stranded silencer is an RNA molecule that is produced from a promoter region of a plant endogene target (i.e., a nucleic acid silencer molecule) that is in an antisense orientation with respect to the plant operable promoter in the nucleic acid construct. In another embodiment, the single-stranded silencer is an RNA molecule that is produced from a promoter region of a plant endogene target (i.e., a nucleic acid silencer molecule) that is in a sense orientation with respect to the plant operable promoter in the nucleic acid construct. In each of these single-stranded silencer embodiments, the construct, nucleic acid silencer molecule and single-stranded silencers are in the absence of inverted repeat structures, i.e., no inverted repeat structures or inverted repeats are present in the nucleic acid construct and products produced from it. Alternatively, in another embodiment, the nucleic acid silencer molecule is an inverted repeat silencer, and is an RNA molecule that is produced from a promoter region of a plant endogene target, wherein the RNA molecule is provided in duplicate and arranged in an inverted configuration in the nucleic acid construct. In one embodiment, the duplicate copies of the target sequence in the inverted repeat structure are separated by a spacer. In one embodiment, the spacer contains an intron functional in a plant cell. In another embodiment, the spacer is a fragment from a soybean 7S promoter. However, it is contemplated that the spacer sequence is not limited to these features and may be any sequence suitable for allowing the inverted repeat sequences to hybridize. Such a spacer sequence is exemplified by SEQ ID NO: 55. In some embodiments, expression of the nucleic acid silencer molecule produces an initial single stranded RNA. This single stranded RNA may be converted to a double stranded RNA by cellular mechanisms or as a result of the inverted repeat structure.

In one embodiment, the promoter region comprises nucleotides upstream of the transcription start site of the target gene. In another embodiment, the promoter region comprises nucleotides upstream of the transcription start site and nucleotides downstream of the transcription start site of the target gene. In some embodiments, the promoter region comprises about 300 nucleotides to about 1500 nucleotides. In other embodiments, the promoter region comprises about 400 nucleotides to about 1200 nucleotides. In additional embodiments, the promoter region comprises about 425 nucleotides to about 1100 nucleotides. In further embodiments, the promoter region comprises about 425 nucleotides to about 1075 nucleotides.

Any promoter that is operable in a plant may be used in the nucleic acid construct. In some embodiments, the promoter is a single copy of a plant operable promoter, including those described herein. In other embodiments, the promoter is a double copy of a plant operable promoter to make a homologous double promoter. In further embodiments, the promoter is a combination of two different promoters to make a heterologous double promoter. In one embodiment, the plant operable promoter is a double 35S CMV promoter. In an additional embodiment, the plant operable promoter is a single 35S CMV promoter. The sequence of the double 35S CMV promoter is set forth in SEQ ID NO: 54. The nucleic acid construct may further comprise sequences to enable cloning of the nucleic acid construct or sequences that will facilitate splicing. In one embodiment, the additional sequence may be a 3′ sequence that is operable in plants. In another embodiment, the 3′ sequence is derived from TRV2 (tobacco rattle virus 2) which is positioned downstream of the nucleic acid silencer molecule. The sequence of the TRV2 3′ sequence is set forth in SEQ ID NO: 53.

The nucleic acid construct may also comprise plant operable 3′ regulatory sequences. In one embodiment, the plant operable 3′ regulatory sequence is a polyA addition sequence. In another embodiment the polyA addition sequence is a NOS polyA sequence.

In a second aspect, the present invention provides a transgenic plant cell comprising the nucleic acid construct. In one embodiment, the nucleic acid construct is stably integrated into the genome of the transgenic plant cell. The transgenic plant cell is prepared by transfecting a plant cell with a nucleic acid construct using methods well known in the art including, but not limited to, those described herein. Plant cells of a wide variety of plant species can be transfected with a nucleic acid construct of the present invention. A plant cell containing the nucleic acid construct is selected in accordance with conventional techniques including, but not limited to, those described herein. The plant cell is grown under conditions suitable for the expression of the nucleic acid in the transfected plant cell using growth conditions well known in the art.

The present invention may be used for transfecting plant cells of a wide variety of plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setara italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats (Avena sativa), barley (Hordeum vulgare), switchgrass (Panicum virgatum), vegetables, ornamentals, and conifers. See U.S. Pat. No. 7,763,773 for a list of additional plant species that can be used in accordance with the present invention.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulchernima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesil); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

In a third aspect, the present invention provides a transgenic plant comprising the nucleic acid construct. In one embodiment, the nucleic acid construct is stably integrated into the genome of the transgenic plant. Transgenic plants are regenerated from transgenic plant cells described herein using conventional techniques well known to the skilled artisan using various pathways, including somatic embryogenesis and organogenesis. Transformed plant cells which are derived by plant transformation techniques, including those discussed above, can be cultured to regenerate a whole plant which possesses the transformed genotype, and thus the desired phenotype. Such regeneration techniques generally rely on manipulation of certain phytohormones in a tissue culture growth medium, typically relying on a marker which has been introduced together with the desired nucleotide sequences. See, for example, U.S. Pat. No. 7,763,773, U.S. Patent Application Publication No. 2010/0199371 and International Published Application No. WO 2008/094127 and references cited therein. The transgenic plant is grown under conditions suitable for the expression of the nucleic acid in the transfected plant using growth conditions well known in the art.

In a fourth aspect, the present invention provides a method of reducing endogenous gene expression in plants, plant tissues or plant cells via transcriptional gene silencing. In one embodiment, the method comprises transfecting a plant cell with the nucleic acid construct to produce a transgenic plant cell as described herein. The method further comprises expressing the nucleic acid silencer molecule in the transgenic plant cell as described herein. The expressed nucleic acid silencer molecule, i.e., an RNA single stranded sense silencer, an RNA single stranded antisense silencer or an RNA IR silencer described herein, is cleaved in the transgenic plant cell to produce one or more small RNAs (sRNAs) that induces transcriptional gene silencing to reduce expression of the target gene of interest. In some embodiments, expression of the nucleic acid silencer molecule produces an initial single stranded RNA. This single stranded RNA may be converted to a double stranded RNA before processing to produce sRNAs by cellular mechanisms or as a result of the inverted repeat structure. The method may optionally include preparing a nucleic acid construct encoding a nucleic acid as described herein. In another embodiment, the method comprises regenerating a transgenic plant from the transgenic plant cell. In this embodiment, the nucleic acid is expressed in the transgenic plant. The expressed nucleic acid is cleaved in the transgenic plant cell to produce one or more sRNAs that induces transcriptional gene silencing to reduce expression of the target gene of interest.

The nucleic acid molecule encoding a single strand silencer or an IR silencer and that is inserted into plants (nucleic acid molecule of interest) in accordance with the present invention is not critical to the transformation process. Generally the nucleic acid molecule of interest that is introduced into a plant is part of a construct as described herein. The construct typically includes regulatory regions operatively linked to the 5′ side of the nucleic acid molecule of interest and/or to the 3′ side of the nucleic acid molecule of interest. A cassette containing all of these elements is also referred to herein as an expression cassette. The expression cassettes may additionally contain 5′ leader sequences in the expression cassette construct. The regulatory regions (i.e., promoters, transcriptional regulatory regions, and transcriptional termination regions) may be native/analogous to the host cell or to each other. Alternatively, the regulatory regions may be heterologous to the host cell or to each other. See, U.S. Pat. Nos. 7,205,453 and 7,763,773, and U.S. Patent Application Publication Nos. 2006/0218670, 2006/0248616 and 20090100536, and the references cited therein.

The nucleic acid molecule of interest that is under control of a plant operable promoter may be any nucleic acid molecule as defined herein and may be used to alter any characteristic or trait of a plant species into which it is introduced through the mechanism of transcriptional gene silencing in order to downregulate the target gene. The target gene may encode a regulatory protein, such as a transcription factor and the like, a binding or interacting protein, or a protein that alters a phenotypic trait of a transgenic plant cell or a transgenic plant. Downregulation of a target gene may enhance, alter or otherwise modify a trait of the plant, such as an agronomic trait. The agronomic trait may relate to plant morphology, physiology, growth and development, yield, nutrition, disease or pest resistance, or environmental or chemical tolerance. In some aspects, the trait is selected from group of traits consisting of water use efficiency, temperature tolerance, yield, nitrogen use efficiency, seed protein, seed oil and biomass. Yield may include increased yield under non-stress conditions and increased yield under environmental stress conditions. Stress conditions may include, for example, drought, shade, fungal disease, viral disease, bacterial disease, insect infestation, nematode infestation, extreme temperature exposure (cold or hot), osmotic stress, reduced nitrogen nutrient availability, reduced phosphorus nutrient availability and high plant density. In some embodiments, the nucleic acid molecule of interest may be used to modify metabolic pathways, such as fatty acid biosynthesis or lipid biosynthesis pathways in seeds, or to modify resistance to pathogens in plants.

Generally, the expression cassette may additionally comprise a selectable marker gene for the selection of transformed cells. Selectable marker genes are utilized for the selection of transformed cells or tissues. Usually, the plant selectable marker gene will encode antibiotic resistance, with suitable genes including at least one set of genes coding for resistance to the antibiotic spectinomycin, the streptomycin phosphotransferase (spt) gene coding for streptomycin resistance, the neomycin phosphotransferase (nptII) gene encoding kanamycin or geneticin resistance, the hygromycin phosphotransferase (hpt or aphiv) gene encoding resistance to hygromycin, acetolactate synthase (als) genes. Alternatively, the plant selectable marker gene will encode herbicide resistance such as resistance to the sulfonylurea-type herbicides, glufosinate, glyphosate, ammonium, bromoxynil, imidazolinones, and 2,4-dichlorophenoxyacetate (2,4-D), including genes coding for resistance to herbicides which act to inhibit the action of glutamine synthase such as phosphinothricin or basta (e.g., the bar gene). See generally, International Publication No. WO 02/36782, U.S. Pat. Nos. 7,205,453 and 7,763,773, and U.S. Patent Application Publication Nos. 2006/0218670, 2006/0248616, 2007/0143880 and 20090100536, and the references cited therein. See also, Jefferson et al. (1991); De Wet et al. (1987); Goff et al. (1990); Kain et al. (1995) and Chiu et al. (1996). This list of selectable marker genes is not meant to be limiting. Any selectable marker gene can be used. The selectable marker gene is also under control of a promoter operable in the plant species to be transformed. Such promoters include those described in International Publication No. WO 2008/094127 and the references cited therein. See also, U.S. Patent Application Publication Nos. 2008/0313773 and 2010/0199371 for an exemplification of additional markers that can be used in accordance with the present invention.

A number of promoters can be used in the practice of the invention. The promoters can be selected based on the desired outcome. That is, the nucleic acids can be combined with constitutive, tissue-preferred, or other promoters for expression in the host cell of interest. Such constitutive promoters include, for example, the core promoter of the Rsyn7 (WO 99/48338 and U.S. Pat. No. 6,072,050); the core CaMV35S promoter (Odell et al., 1985); rice actin (McElroy et al., 1990); ubiquitin (Christensen and Quail, 1989; Christensen et al., 1992); pEMU (Last et al., 1991); MAS (Velten et al., 1984); ALS promoter (U.S. Pat. No. 5,659,026), and the like. Other constitutive promoters include, for example, those disclosed in U.S. Pat. Nos. 5,608,149; 5,608,144; 5,604,121; 5,569,597; 5,466,785; 5,399,680; 5,268,463; and 5,608,142.

Other promoters include inducible promoters. Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners Pathogen-inducible promoters include those from pathogenesis-related proteins (PR proteins), which are induced following infection by a pathogen; e.g., PR proteins, SAR proteins, beta-1,3-glucanase, chitinase, etc. Other promoters include those that are expressed locally at or near the site of pathogen infection. In further embodiments, the promoter may be a wound-inducible promoter. In other embodiments, chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. In addition, tissue-preferred promoters can be utilized to target enhanced expression of a polynucleotide of interest within a particular plant tissue. Each of these promoters is described in U.S. Pat. Nos. 6,506,962, 6,575,814, 6,972,349 and 7,301,069 and in U.S. Patent Application Publication Nos. 2007/0061917 and 2007/0143880. See also, U.S. Patent Application Publication Nos. 2008/0313773 and 2010/0199371 for an exemplification of additional promoters that can be used in accordance with the present invention. Other promoters known to the skilled artisan to be useful in plants, can also be used in the present invention.

Promoters for use in the current invention may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (U.S. Patent Application Publication No. 2006/0156439), the maize ROOTMET2 promoter (International Publication No. WO 05/063998), the CR1BIO promoter (International Publication No. WO 06/055487), the CRWAQ81 promoter (International Publication No. WO 05/035770) and the maize ZRP2.47 promoter (NCBI accession number: U38790; GI No. 1063664). In some embodiments, the promoter that is used is a double promoter, for example, a double CaMV 35S promoter. Double promoters of any of the promoters disclosed herein, as well as other promoters known to the skilled artisan to be useful in plants, can be used in the present invention.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g. transitions and transversions may be involved.

Once a nucleic acid has been cloned into an expression vector, it may be introduced into a plant cell using conventional transformation (or transfection) procedures. The term “plant cell” is intended to encompass any cell derived from a plant including undifferentiated tissues such as callus and suspension cultures, as well as plant seeds, pollen or plant embryos. Plant tissues suitable for transformation include leaf tissues, root tissues, meristems, protoplasts, hypocotyls, cotyledons, scutellum, shoot apex, root, immature embryo, pollen, and anther. “Transformation” means the directed modification of the genome of a cell by the external application of recombinant DNA from another cell of different genotype, leading to its uptake and integration into the subject cell's genome. In this manner, genetically modified plants, plant cells, plant tissue, seed, and the like can be obtained.

DNA constructs in accordance with the present invention can be used to transform any plant. The constructs may be introduced into the genome of the desired plant host by a variety of conventional techniques. Techniques for transforming a wide variety of higher plant species are well known and described in the technical and scientific literature. Transformation protocols may vary depending on the type of plant or plant cell, i.e., monocot or dicot, targeted for transformation, as is well known to the skilled artisan. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Thus, any method, which provides for effective transformation/transfection may be employed. See, for example, U.S. Pat. Nos. 7,241,937, 7,273,966 and 7,291,765 and U.S. Patent Application Publication Nos. 2007/0231905 and 2008/0010704 and references cited therein. See also, International Published Application Nos. WO 2005/103271 and WO 2008/094127 and references cited therein. See also, U.S. Patent Application Publication Nos. 2008/0313773 and 2010/0199371 for an exemplification of transformation protocols for a variety of plant species that can be used in accordance with the present invention.

Transformed plant cells which are derived by any of the above transformation techniques can be cultured to regenerate a whole plant which possesses the transformed genotype and thus the desired phenotype, e.g., a transgenic plant. A “transgenic plant” is a plant into which foreign DNA has been introduced. A “transgenic plant” encompasses all descendants, hybrids, and crosses thereof, whether reproduced sexually or asexually, and which continue to harbor the foreign DNA. Regeneration techniques rely on manipulation of certain phytohormones in a tissue culture growth medium, typically relying on a biocide and/or herbicide marker which has been introduced together with the desired nucleotide sequences. See for example, International Published Application No. WO 2008/094127 and references cited therein and U.S. Patent Application Publication No. 2010/0199371.

The foregoing methods for transformation are typically used for producing a transgenic variety in which the expression cassette is stably incorporated. After the expression cassette is stably incorporated in transgenic plants, it can be transferred to other plants by sexual crossing. In one embodiment, the transgenic variety could then be crossed, with another (non-transformed or transformed) variety, in order to produce a new transgenic variety. Alternatively, a genetic trait which has been engineered into a particular cotton line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties which do not contain that gene. As used herein, “crossing” can refer to a simple X by Y cross, or the process of backcrossing, depending on the context. Any of a number of standard breeding techniques can be used, depending upon the species to be crossed.

Once transgenic plants of this type are produced, the plants themselves can be cultivated in accordance with conventional procedures. Transgenic seeds can, of course, be recovered from the transgenic plants. These seeds can then be planted in the soil and cultivated using conventional procedures to produce transgenic plants. The cultivated transgenic plants will express the nucleic acid as described herein and it will be cleaved to produce sRNAs.

In a fifth aspect, the present invention provides nucleic acid constructs and methods to identify and obtain other TGS silencers from promoter regions of endogenes. According to this aspect, the nucleic acid construct is one that is suitable for transformation of a plant species for which it is desired to identify a TGS silencer. In one embodiment, the nucleic acid construct may be included in a vector. In some embodiments, the vector is suitable for Agrobacterium-mediated transformation. In other embodiments, the vector may be suitable for biolistic-mediated transformation. Other suitable vectors for plant transformations are well known to the skilled artisan. In another embodiment, the nucleic acid construct may be used directly for the transformation of a plant according to techniques well known to the skilled artisan. The nucleic acid construct comprises a plant operable promoter operatively linked to a putative nucleic acid silencer molecule operatively linked to plant operable 3′ regulatory region. In one embodiment, the putative nucleic acid silencer molecule comprises a promoter region of a plant endogene target which is to be tested for transcriptional gene silencing. In some embodiments, the putative nucleic acid silencer molecule is in a sense orientation with respect to the plant operable promoter as described herein. In other embodiments the putative nucleic acid silencer molecule is in an antisense orientation with the respect to the plant operable promoter. In further embodiments, the nucleic acid silencer molecule may contain inverted repeats or inverted repeat structures as described herein. In one embodiment, the plant operable promoter is a single promoter, a double homologous promoter or a double heterologous promoter. In one embodiment, the plant operable promoter is a single or a double 35S CMV promoter. In one embodiment, the 3′ sequence is a TRV2 3′ sequence. In an additional embodiment, the 3′ regulatory region is a polyA addition sequence. In one embodiment, the polyA sequence is a NOS poly A sequence. The testing for silencing may be carried out according to methods well known in the art. These methods include, but are not limited to, RT-PCR, PCR, northern blot analysis, immunological assay and enzymatic assay. In one embodiment, the methylation state of the target promoter may be assayed. In yet a further embodiment, the methylation state of the target promoter may be assayed by McrBc enzymatic digestion. In yet another embodiment, the state of chromatin modification is tested by immunological assays utilizing antibodies directed to sites of histone methylation or acetylation.

Further according to this aspect, suitable TGS silencers are identified by a method which comprises the steps of preparing a nucleic acid construct comprising a putative nucleic acid silencer molecule of an endogene of interest as described herein, transforming a cell or tissue of a plant species of interest with the nucleic acid construct and determining whether the putative nucleic acid silencer molecule is processed by the transformed cell or tissue of the plant species to produce silencing of the endogene of interest. If the endogene is silenced, then the putative nucleic acid silencer molecule of the endogene of interest is identified as a TGS silencer. In one embodiment, the determination is made by culturing the transformed plant cell for expression of the putative nucleic acid silencer molecule and testing for transcriptional gene silencing in the cultured transformed plant cell or tissue. In another embodiment, the determination is made by regenerating a transformed plant from the transformed plant cell or tissue and testing for transcriptional gene silencing in the transformed plant. The regeneration of transformed plants is performed according to techniques well known to the skilled artisan. The testing for silencing may be carried out according to methods well known in the art. These methods include, but are not limited to, RT-PCR, PCR, northern blot analysis, immunological assay and enzymatic assay. In one embodiment, the methylation state of the target promoter may be assayed. In yet a further embodiment, the methylation state of the target promoter may be assayed by McrBc enzymatic digestion. In yet another embodiment, the state of chromatin modification is tested by immunological assays utilizing antibodies directed to sites of histone methylation or acetylation.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Green and Sambrook, 2012, Molecular Cloning, 4th Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Russell, 1984, Molecular biology of plants: a laboratory course manual (Cold Spring Harbor Laboratory Press, Cold Spring Harbor, N.Y.); Anand, Techniques for the Analysis of Complex Genomes, (Academic Press, New York, 1992); Guthrie and Fink, Guide to Yeast Genetics and Molecular Biology (Academic Press, New York, 1991); Harlow and Lane, 1988, Antibodies, (Cold Spring Harbor Laboratory Press, Cold Spring Harbor, N.Y.); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Fire et al., RNA Interference Technology: From Basic Science to Drug Development, Cambridge University Press, Cambridge, 2005; Schepers, RNA Interference in Practice, Wiley-VCH, 2005; Engelke, RNA Interference (RNAi): The Nuts & Bolts of siRNA Technology, DNA Press, 2003; Gott, RNA Interference, Editing, and Modification: Methods and Protocols (Methods in Molecular Biology), Human Press, Totowa, N.J., 2004; Sohail, Gene Silencing by RNA Interference: Technology and Application, CRC, 2004.

### EXAMPLES

The present invention is described by reference to the following Examples, which is offered by way of illustration and is not intended to limit the invention in any manner. Standard techniques well known in the art or the techniques specifically described below were utilized.

### Example 1

### Materials and Methods

Constructs:

Using standard PCR and cloning techniques, a 475 bp genomic DNA fragment from promoter region (−483 to −9, refers to transcription start site as +1) (SEQ ID NO:1) of AT1G80080 (TMM) was cloned into a binary plasmid pBAV3. The latter plasmid was derived from pBA002 (Kost et al., 1998) by replacing the NOS polyA addition sequence near the right border of T-DNA with a fragment from pTRV2 and replacing the 35S promoter with a duplicated 35S promoter from pBCO-DC-CFP (Wu et al., 2010). The TMM promoter fragment was inserted downstream of the double 35S promoter either in the sense (S silencer) or the antisense (AS silencer) orientation with respect to the TMM coding sequence. To obtain an inverted repeat structure, the TMM promoter DNA fragment was cloned first into vector pSRS, which is derived from pSK-int (Guo et al., 2003) by replacing the intron with a soybean promoter sequence (Mette et al., 2000). The inverted repeat structure (IR silencer) was the cloned into pBAV3 under the control of a double 35S promoter. The S-, AS- and IR-silencer targeting on the TMM promoter were also cloned into a conventional pBA002 mRNA expression vector (Kost et al., 1998) to generate another series of silencers with a NOS polyA addition sequence. By a similar strategy, the silencer constructs for FHY1 (−449 to −1) (SEQ ID NO:2), HFR1 (−1025 to −1) (SEQ ID NO:3), and PhyB (−945 to +112) (SEQ ID NO:4) were generated.

Plant Materials and Transformation:

We used Arabidopsis thaliana Columbia (Col-0) ecotype as the wild type (WT) in this study. All A. thaliana mutants have been described: rdr2-2 (SALK_{—}059661, (Herr et al., 2005)), sgs2-1/rdr6 (Elmayan et al., 1998), dcl3-1 (SALK_{—}005512, (Xie et al., 2004)), ago4-2 (Agorio and Vera, 2007), nrpd1a-3 (SALK_{—}128428, (Herr et al., 2005)), nrpd1b-1/nrpe1-1 (SALK_{—}029919, (Pontier et al., 2005)), ddc (drm1/crm2/cmt3 triple mutant, (Zhang et al., 2006b)), PhyB-9 (Reed et al., 1993), hfr1-201 (Soh et al., 2000), and fhy1-3 (Zeidler et al., 2004). All mutants are in the Columbia (Col-0) ecotype background.

Plants were grown in a growth chamber under long-day (16 hour of light/8 hour of dark) condition illuminated by cool-white fluorescent light (100 μmol/m²/s) until flowering. Standard floral dip transformation was performed according to Zhang et al. (Zhang et al., 2006a).

Tmm-Phenotype Observation:

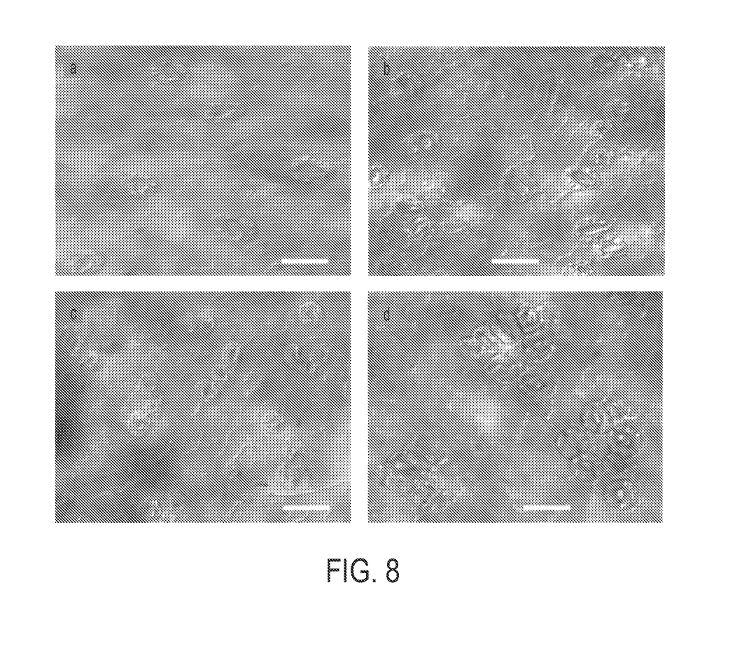

After harvest, T1 seedlings were screened on BASTA agar plates. Morphological phenotype was scored under a microscope. For each independent transgenic line, penetration rate was calculated from 40-80 randomly picked 3-week-old seedlings. We classified the tmm-phenotype roughly into 3 categories: weak, intermediate, and strong according to the frequency and number of clustered stomata (FIGS. 8a-8d). When plants from a transform event showed predominantly two clustered stomata and occasionally three clustered stomata, we scored this as a weak phenotype. Transgenic plants displayed an intermediated tmm-phenotype if three clustered stomata were frequently observed. Transgenic plants with a strong tmm-phenotype showed more than four clustered stomata and usually single stomata could not be observed (FIGS. 8a-8d).

DNA Methylation Analysis:

Arabidopsis genomic DNA was extracted from 2-week-old seedlings by DNeasy Plant Mini Kit (Qiagen, Cat. No. 69104).

Sequencing of bisulphate-treated DNA samples was performed as follows. Bisulfite DNA conversion was performed by using 1 μg genomic DNA and EpiTech Bisulfite Kit (Qiagen, Cat. No. 59104) following the manufacturer's protocol. PCR was performed using primers outside of the targeted region and designed for single strand methylation detection. Primers used in connection with the Examples are set forth in Table 1. PCR products were then cloned into pCR2.1 using TA cloning kit (Invitrogen, Cat. No. K2020-40). For each genotype, at least 20 independent clones were sequenced using M13R primer. Data were analyzed by Cymate (Hetzl et al, 2007).

TABLE 1
Primers

| NAME | SEQUENCE (5′-3′) (SEQ ID NO:) | DESCRIPTION |
|---|---|---|
| pBANOSF | GGGAGCTCGTAATCAGGATCCTAGGTGTTTCCTG (5) | NOS terminator |
| pBANOSR | CAAACCGCCTCTCCCCGGGCGTTGG (6) | knock out |
| pBAV3F | CGCGTCTGGATCCCCGGGCATGTCCC (7) | TRV2 3′ |
| pBAV3R | GTGCCTAGGGTTCTCCCGTTTCG (8) | sequence cloning |
| pBAD35sF | CCGCTCATGATCTTGATCCCCTGCGCC (9) | double 35s |
| pBAD35sR | GTAAAAATACTCTAGACCGGCCTCTC (10) | cloning |
| pTMMASF | GACACGTATTAGATCTGTTGCTCCATGGGCAT (11) | pTMM AS |
| pTMMASR | GGACTAGTTTCGAAATTGTCAGTGTG (12) | silencer cloning |
| pTMMSF | GTTCCTAGGTGTTGCTCCATGGGCA (13) | pTMM S |
| pTMMSR | GAATGAGATCTTCGAAATTGTCAGTGT (14) | silencer cloning |
| pTMMBIF3 | AAAATAATGATTTTAAGGTT (15) | pTMM bisulfite |
| pTMMBIR2 | AATTCATATCRTRCCATTTC (16) | cloning |
| UniBIF2 | AAGYAAGTGGATTGATGTGATAT (17) | TMM Silencer |
| UniBIR3 | CCTAAAACTTCARACACRRATCTA (18) | bisulfite cloning |
| TMMF | GTGGAGGATGAGGAGAAAGCTGA (19) | TMM transcripts |
| TMMR | ATAGGTGCTGGACCGTACCGGAAG (20) | & ChIP qPCR |
| pTMM5′F | CTAATCTTTGTTTTGATGCTTTC (21) | ChIP qPCR |
| pTMM5′R | CTGTATTTGTTTTTGTAACAAACC (22) | |
| pTMM3′F | GAGTTATCCCGTGAAAGTCA (23) | ChIP qPCR |
| pTMM3′R | GAATATTTGGCGGAAGAATTCA (24) | |
| McrBc5′F | CATCAGACGGCTGTACGACTGC (25) | McrBc digest 5′ |
| McrBc5′R | CTGTATTTGTTTTTGTAACAAACC (26) | qPCR |
| McrBc3′F | CTCGAGTTATCCCGTGAAAGT (27) | McrBc digest 3′ |
| McrBc3′R | CGGAGGTGATCACAGTGAAAGATC (28) | qPCR |
| pTMMT7S1F | TAATACGACTCACTATAGGGGTAAGATGACACGTATTACATG | pTMM sense |
| | (29) | RNA probe A |
| pTMMS1R | TTATTTGGGCTGAGCCCATT (30) | |
| pTMMT7S2F | TAATACGACTCACTATAGGGAAAAAAAGTAAACGATAAGGA | pTMM sense |
| | G (31) | RNA probe B |
| pTMMS2R | GTGTGAATGCGGTTTAGGTTCGAA (32) | |
| pTMMT7AS1F | TAATACGACTCACTATAGGGGGTTTAGGTTCGAAATTGTC | pTMM antisense |
| | (33) | RNA probe A |
| pTMMAS1R | AAAAAAAGTAAACGATAAGGAG (34) | |
| pTMMT7AS2F | TAATACGACTCACTATAGGGTTATTTGGGCTGAGCCCATT | pTMM antisense |
| | (35) | RNA probe B |
| pTMMAS2R | GTAAGATGACACGTATTACATG (36) | |
| pHFR1F | GGTCTCGAGATCTTTGGATAAAACCCTAAATGAATTCT (37) | HFR1 silencer |
| pHFR1R | CATGGATCCCACTAGTATTGTGCCACGTTTCAAAG (38) | cloning |
| HFR1QF | CGTATCCAGGTCTTAAGTAGTGATGATGAAT (39) | HFR1 transcripts |
| HFR1QR | CTTGTGACAATTAGGTACGAGTTGCTGTAG (40) | qPCR |
| pFHY1SF | AGCCTCGAGAATTTTTGTAAAAAAAATCCAAA (41) | sense FHY1 |
| pFHY1SR | AGCGGATCCGAAAATCTGGAAACTGCGTAACT (42) | silencer cloning |
| pFHY1ASF | AGCCTCGAGGAAAATCTGGAAACTGCGTAACT (43) | antisense FHY1 |
| pFHY1ASR | AGCGGATCCAATTTTTGTAAAAAAAATCCAAA (44) | silencer cloning |
| HSO3_F | CAAATTRTCCAAAAAAATAATTACC (45) | pFHY1 bisulfite |
| HSO3_R | GAGAGTTYAAAGATAATTGTTTTT (46) | cloning |
| FHY1_QF | TGGAAGATGGACTGTTAACCAAGATG (47) | FHY1 transcripts |
| FHY1_QR | TGAGTCTCAACATCATTTTGTTACAGC (48) | qPCR |
| pPhyBSF | GGTCTCGAGAAATTAGTCATTCAACAAAAGA (49) | sense PhyB |
| pPhyBSR | GTAGAGCTCACTAGTTATCGGAGACGAATTC (50) | silencer cloning |
| pPhyBASF | CACCGAGCTCACTAGTTATCGGAGACGAATTCTG (51) | antisense PhyB |
| pPhyBASR | CTCGGATCCAAATTAGTCATTCAACAAAAGA (52) | silencer cloning |

McrBc digestion was performed as follows. 250 ng genomic DNA were digested in a total volume of 20 μl by McrBc (New England Lab, Cat no M0272S) following the manufacturer's instruction. Treated and untreated control samples were then used as template in real time PCR reactions.

Chromatin Immunoprecipitation (CHIP):

Three grams of 2-week-old seedlings were used in immunoprecipitation experiments as described by Gendrel (Gendrel et al. 2005) with minor modification. Cross-linked chromatin pellets resuspended in nuclei lysis buffer were sonicated in a Bioruptor (Bioruptor UCD 200, Diagenode) for 10 min at the max level. Samples were sonicated for periods of 30 sec with 30 s interval in between treatment. Histone H3 trimethyl Lys4 (K4me3) antibody were from Active Motif (Cat. No. 39159). Histone H3 Acetylation, Histone H3 trimethyl Lys9 (K9me3), and Histone H3 trimethyl Lys27 (K27me3) antibodies were from Milipore (Cat. No. 06-599, 07-442, 07-449).

RNA Isolation and RT-PCR:

RNA was extracted from 2 weeks old seedlings by RNeasy Plant Mini Kit (Qiagen, Cat. No. 74904) following the manufacturer's instructions. cDNA synthesis was performed by using Superscript III First strand synthesis system (Invitrogen, Cat. No. 18080051) following the manufacturer's instructions.

Real-Time PCR:

Real-time PCR was performed using SYBR Premix Ex Taq (Takara, Cat. No. RR401A) in a Biorad CFX96 realtime PCR system. ACTIN2 was used as an internal control. The primers used are set forth in Table 1. Real-time quantitative PCR was repeated with two to four biological replicates, and each sample was assayed in triplicate by PCR. Error bars in each graph indicate SD of three technical repetitions.

Small RNA Analysis:

Total RNA was extracted from 2-week-old seedlings by Trizol Reagent (Invitrogen, Cat. No. 15596-026) following the manufacturer's instructions. Small RNA libraries were constructed by TruSeq Small RNA Sample Prep Kit (Illumina, Cat. No. RS-200-0024) following the manufacturer's instructions. Briefly, 1 μg total RNA or purified small RNA was ligated with 3′ and 5′ adaptors and used as a template for RT-PCR. After PCR amplification, 6 μl of each sample were pooled and separated on a 6% polyacrylamide gel. Sequences were determined by Illunina HiSeq in the genomic center in Rockefeller University. The sRNA sequencing data is available at the Gene Expression Omnibus. Adaptor sequence was trimmed by local Perl script and only reads longer than 15 nt were used for further analysis. All retained reads were mapped to the Arabidopsis genome (TAIR 9 version) by C program allowing no mismatch.

RNA gel blots were analyzed using 10 μg of total RNA per lane. RNA was separated by 17% PAGE/8 M Urea/0.5×TBE buffer. The gel was electroblotted to Hybond N+ membrane (Amersham, Piscataway, N.J.) and then UV cross-linked. The probes were made by in vitro transcription of four fragments from the candidate promoter region, two from the sense and two from the anti-sense strand. Hybridizations were performed at 42° C. overnight in UltraHyb hybridization solution (Ambion, Austin, Tex.), according to the supplier's direction. After hybridization, membranes were washed in 2×SSC with 0.1% SDS and analyzed using BioMax MS films (Kodak).

Hypocotyl Phenotype Observation:

Hypocotyl phenotypes were assayed as described before (Jang et al., 2007). Briefly, sterilized seeds were sowed on Murashige and Skoog (MS) plates and stratified at 4° C. in the dark for 4 days then exposed to white light for 1 hour before incubating under far-red light for 4 days at 22° C. Lengths of hypocotyls were recorded afterwards.

### Example 2

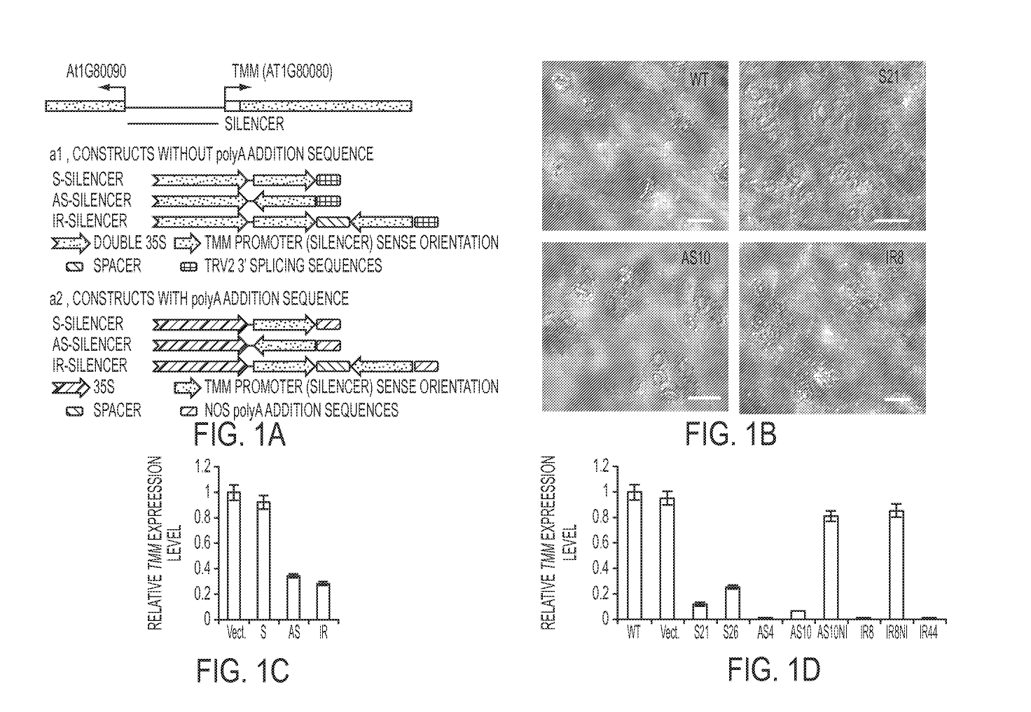

### Single-Stranded Silencer can Induce Tmm-Phenotype as Well as Inverted Repeat Silencer Targeting on Tmm Promoter

Whereas Mette et al. reported success of TGS of a transgene using double-stranded inverted repeat (IR) RNA silencer (Mette et al., 2000) they also mentioned that single-stranded silencers in either the sense (S) or antisense (AS) orientation (expressed separately or together) were ineffective. We compared the effectiveness of these three types of silencer using the Arabidopsis Too Many Mouths (TMM) endogene as a model. We chose TMM because loss-of-function tmm mutants show clustered stomata phenotype on cotyledons, which is convenient to score. We constructed silencers expressing TMM promoter sequence (pTMM −9 to −483) in S, AS (with respect to the coding sequence) and IR configurations. (FIG. 1a). The first series of silencers contained the 3′ end of the TRV2 RNA (FIG. 1a “a1”) whereas the second series contained a NOS polyA addition sequence (FIG. 1a “a2”).

FIGS. 1a-1d show that single-stranded silencers alone as well as inverted double stranded-silencer, were able to induce typical clustering of stomata on cotyledons phenocopying tmm mutants (FIG. 1b). Penetration rates of tmm-phenotype determined from a population of T1 transformants suggested that the silencing efficiency of single-stranded AS silencer was comparable with double-strand IR silencer, whereas the single-stranded S silencer was much less effective (Table 2). Quantitative transcript analysis by qRT-PCR showed that TMM transcript levels reduced significantly in transgenic plants expressing AS and IR silencers (FIGS. 1c and 1d). TMM transcript levels recovered to WT levels when the transgenic silencers were segregated by genetic crosses (AS10NI and IR8NI in FIG. 1d) indicating that the TGS was silencer-dependent.

TABLE 2
Penetration of tmm-Phenotype of T1

| | Phenotype (%) | | |
|---|---|---|---|
| Type of Silencer | Weak | Intermediate | Strong |
| S Silencer | 12.5 | 1.25 | 0 |
| AS Silencer | 15 | 27.5 | 20 |
| IR Silencer | 10 | 22.5 | 40 |

To test whether the TRV2 3′ sequence (SEQ ID NO:53) is necessary for the observed TGS, we used a 35S promoter to express the same pTMM sequence but with a NOS 3′ polyA addition sequence in the pBA002 binary expression vector (Kost et al., 1998). Similar tmm-phenotype were observed in transgenic plants expressing the pBA002-S, -AS, and -IR series of silencers, although with a slightly lower penetration (Table 3). Together, these results show that the effectiveness of single-stranded S and AS silencers in inducing TGS of the TM/VI endogene is not dependent on 3′ end structures.

TABLE 3
Penetration of tmm-Phenotype of pBA002 Constructs T1 Transformants

| | Phenotype (%) | | |
|---|---|---|---|
| Type of Silencer | Weak | Intermediate | Strong |
| S Silencer | 7.5 | 0 | 0 |
| AS Silencer | 7.5 | 25 | 7.5 |
| IR Silencer | 10 | 20 | 32.5 |

### Example 3

### Genomic Insertion Position of Single Strand Silencer

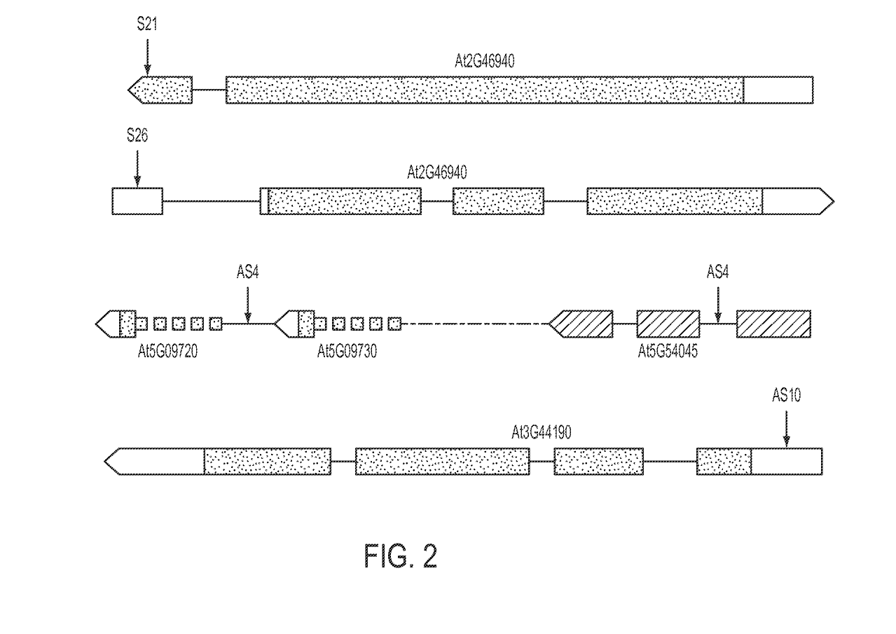

We chose for each construct two independent lines with a nearly 3:1 segregation ratio in the T2 generation for further phenotypic investigations. There was a remote possibility that two copies of the single-stranded silencer might have been inserted into the genome in an inverted orientation. Leaky transcriptional read through could generate RNA complementary to the pTMM sequences and thus forming double-stranded IR RNAs. To rule out this possibility we mapped the insertion positions of several transgenic lines by TAIL-PCR (Liu et al., 1995). The results indicated all the lines tested carried single insertions, except AS4 (AS series, line 4) which harbored two insertions (FIG. 2). The T-DNA of S21 (sense series, line 21) was located near the end of At2g46940, which encodes a protein with unknown function. The T-DNA of S26 landed at the 5′ untranslated region (UTR) of At1g01860, which encodes a glycine cleavage T-protein family protein. One insertion of AS4 was found in the intergenic region between At5g09720, which encodes a magnesium transporter CorA-like family protein and At5g09730, which encodes a protein similar to a beta-xylosidase and the other insertion was located in the first intron of At5g54045, a pseudogene of UF3GT. The T-DNA of AS10 was inserted at the 5′ UTR of At3g44190, which encodes a FAD/NAD(P)-binding oxidoreductase family protein. So far, there was no evidence to show that mutation of these genes had an effect on the expression of TMM or tmm-phenotype.

### Example 4

### Single-Stranded Silencer can Generate Mostly 24nt sRNAs Different from IR-Related sRNAs

It is well documented that TGS can be induced through RNA-directed DNA Methylation (RdDM) with sRNAs, mostly 24-26nt, derived from exo/endo-geneous double-stranded RNAs (Wassenegger et al., 1994; Mette et al., 2000; Hamilton et al., 2002). On the other hand, shorter sRNA species (21-22 nt) mainly mediate degradation of target RNAs with sequence homology resulting in post-transcriptional gene silencing (PTGS) (Hamilton et al., 2002; Vaucheret, 2006).

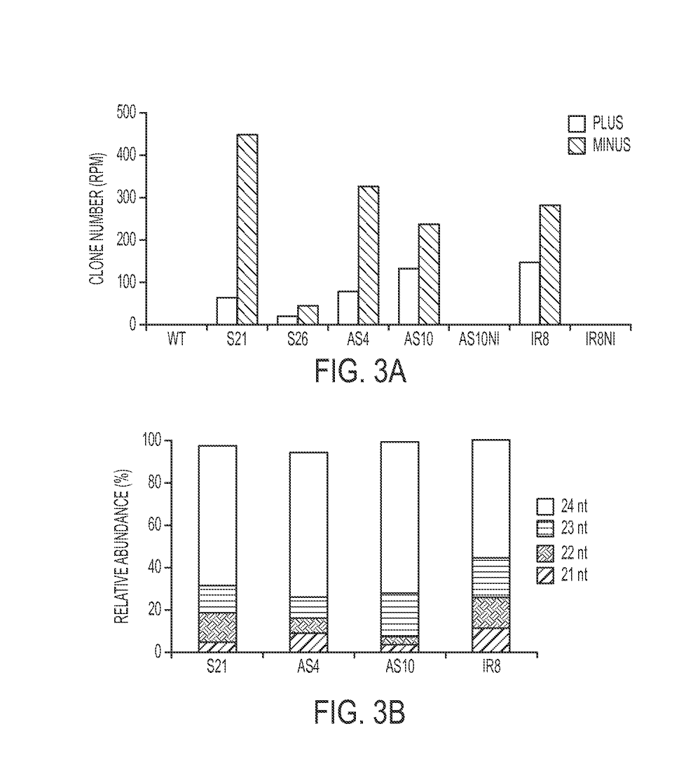

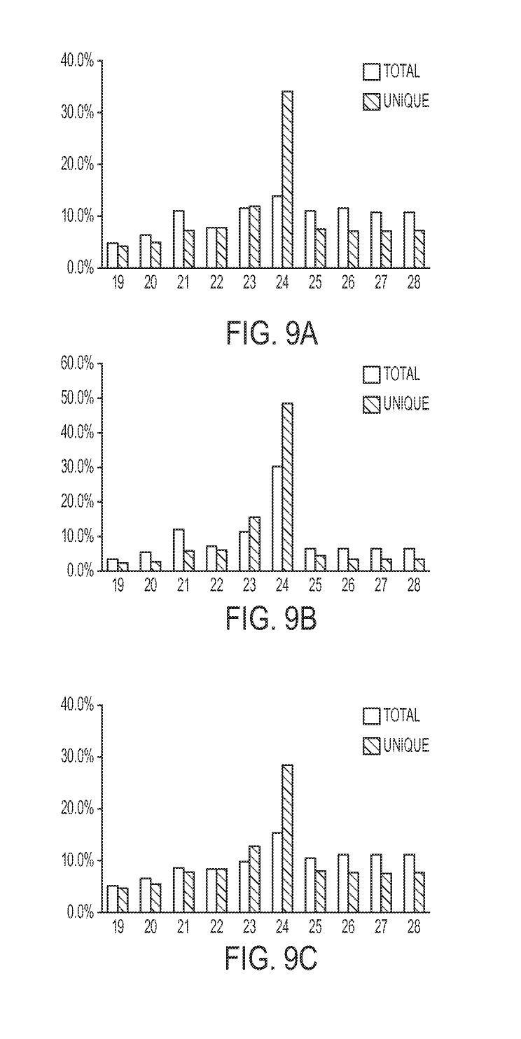

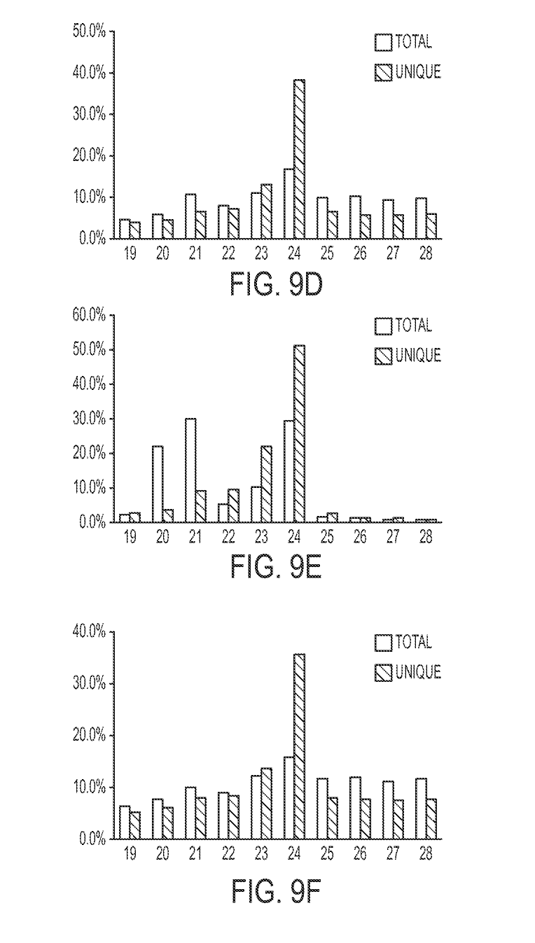

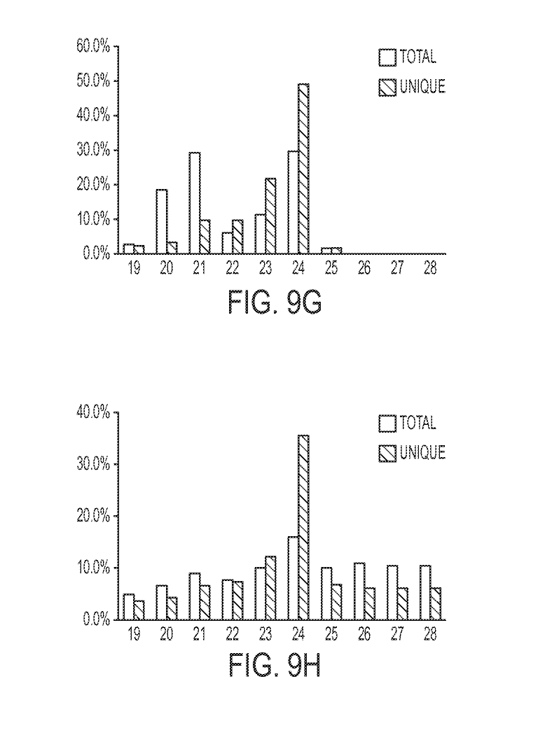

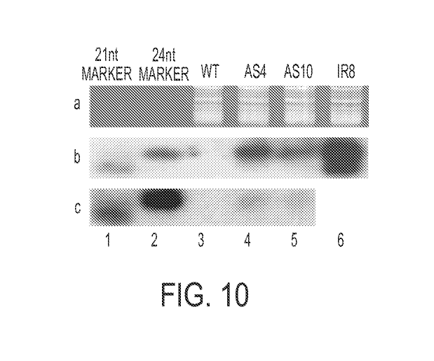

To investigate possible sRNAs linked to TMM silencing by single-stranded and double-stranded silencers, we determined sequences of small RNAs by the Illumina high-throughput sequencing platform. Except for sample S21, of which we obtained 868,571 reads, we generated more than one million reads for each of the other samples. The read number of AS10 and IR8, which were started with purified sRNAs, were one order of magnitude more than other unfractionated samples (Table 4). After removing irrelevant sequences, ˜80% reads could be mapped to the Arabidopsis genome (TAIR 9) (Table 4). For all samples, 24nt sRNAs represented the dominant species amongst the mapped small RNA population (FIG. 9). None of the samples produced any sRNAs corresponding to the TMM coding region. In the untransformed WT sample, we did not recover any sRNAs mapped to the TMM promoter region. By contrast, we indeed recovered an abundance of sRNAs that can be mapped to pTMM in silenced transgenic plants (Table 5, FIG. 3a). Production of sRNAs was dependent on the presence of the silencer. When the silencer transgene was segregated by genetic crosses the sRNAs disappeared (Table 6, AS10NI and IR8NI). We also found that the majority of sRNAs were derived from the minus strand in all 3 categories of silencers (Table 5, FIGS. 10a-10c). Among the sRNAs that can be mapped to the TMM promoter region, almost all were 21-24nt length. For transgenic plants harboring single-strand silencers, more than 70% sRNAs were 24nt in length. This was different from sRNAs derived from double-stranded silencer in which a significant proportion of 21-23nt sRNAs can be detected accounting for ˜45% of all sRNAs (FIG. 3b). Small RNA Northern blot showed a similar pattern (FIGS. 10a-10c) suggesting a different sRNAs biogenesis mechanism generated by single-stranded silencers and by double-stranded silencer.

TABLE 4
Summary of sRNA Sequencing

| | | Clipped | | Mapped | |
|---|---|---|---|---|---|
| Sample | Total | total | unique | total | unique |
| WT | 5,824,035 | 5,511,508 | 721,800 | 4,381,086 | 378,728 |
| S21 | 868,571 | 793,671 | 186,386 | 606,301 | 122,033 |
| S26 | 1,068,652 | 987,181 | 181,343 | 818,493 | 120,431 |
| AS4 | 3,629,865 | 2,627,351 | 448,118 | 2,073,267 | 251,960 |
| AS10 | 29,520,225 | 29,196,326 | 8,749,079 | 19,637,148 | 2,647,203 |
| AS10NI | 3,517,458 | 3,330,102 | 460,923 | 2,749,728 | 262,644 |
| IR8 | 30,510,016 | 30,094,803 | 9,204,333 | 19,740,551 | 2,797,682 |
| IR8NI | 2,075,946 | 1,945,881 | 298,879 | 1,615,622 | 185,094 |

TABLE 5
Result of sRNA Mapping

| | | |
|---|---|---|
| | | Mapped to |
| Total | 21-24nt | pTMM |

| Sample | Mapped | mapped | plus | minus |
|---|---|---|---|---|
| WT | 4,381,086 | 924,984 | 0 | 0 |
| S21 | 606,301 | 190,799 | 12 | 86 |
| S26 | 818,493 | 150,757 | 3 | 7 |
| AS4 | 2,073,267 | 468,597 | 37 | 153 |
| AS10 | 19,637,148 | 14,646,135 | 1,951 | 3,473 |
| AS10NI | 2,749,728 | 591,549 | 0 | 0 |
| IR8 | 19,740,551 | 15,397,037 | 2,260 | 4,357 |
| IR8NI | 1,615,622 | 325,687 | 0 | 0 |

TABLE 6
Penetration of tmm-Phenotype of T1 Transformants in Different
Arabidopsis Mutants

| | Type of Silencer | | |
|---|---|---|---|
| Background | S Silencer | AS Silencer | IR Silencer |
| WT/Col-0 | 13.75 | 62.5 | 72.5 |
| rdr2-2 | 10 | 54 | 68 |
| sgs2-1 | 12.5 | 70 | 75 |
| dcl3-1 | 8 | 46 | 48 |
| ago4-2 | 11.25 | 50 | 55 |
| nrpd1-3 | 14 | 74 | 76 |
| nrpe1-1 | 5 | 15 | 15 |
| ddc | 5 | 10 | 15 |

### Example 5

### AS Silencer-Derived sRNAs not Only Induce De Novo DNA Methylation at Endogenous pTMM but Also at the Silencer Locus

Increased DNA methylation at siRNA-targeted promoter region is generally considered as the hallmark for TGS (Aufsatz et al., 2002) although it is not always linked to the success of silencing (Okano et al., 2008). Mette et al. (Mette et al., 2000) reported single-stranded antisense trigger/silencer can slightly induce DNA methylation at the trigger/silencer transgene but not the targeted promoter. To determine whether the observed sRNAs related to the TMM promoter can actually induce DNA methylation at target promoter and the silencer locus, we performed sequencing of bisulfate-treated DNAs to study the methylation status in detail.

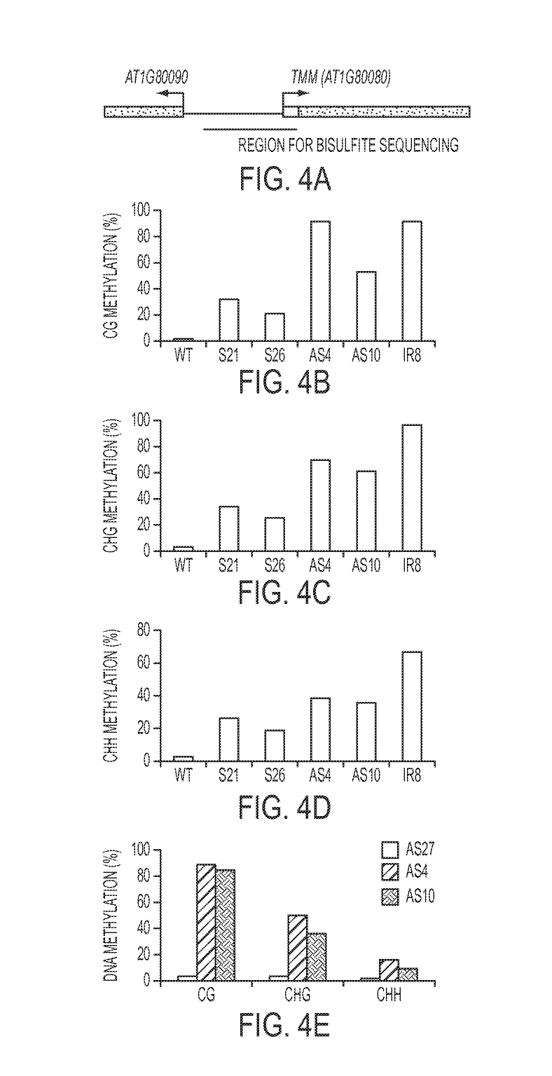

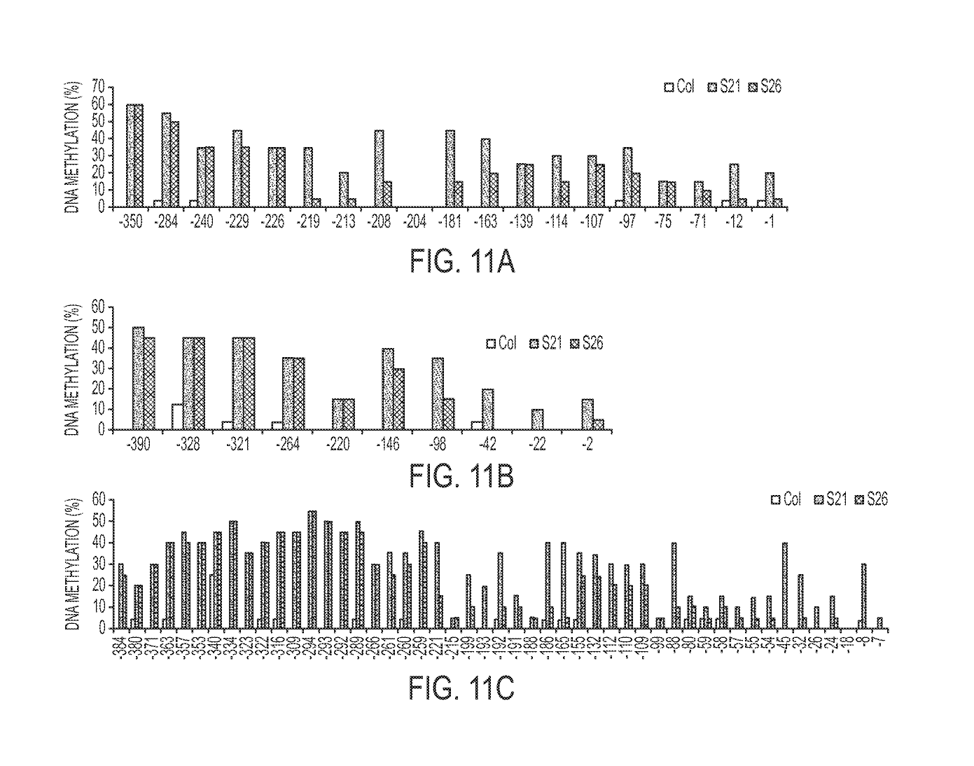

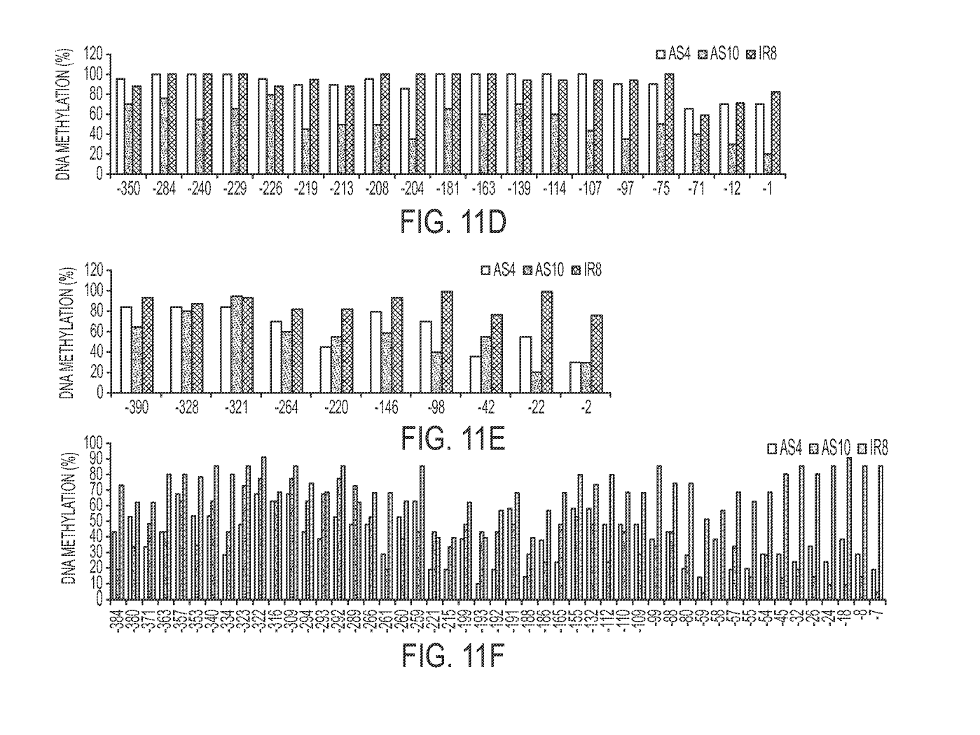

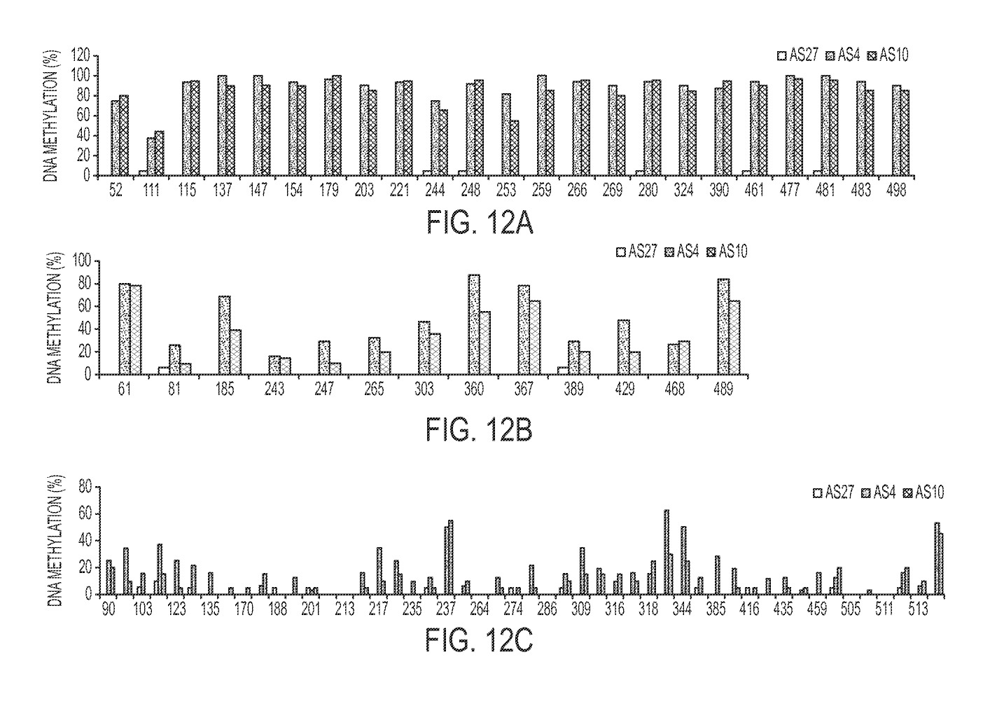

Significant increases in methylation of all three kinds of cytosine, CG, CHG (H means A, C, or T) and CHH was observed at the endogenous TMM promoter region in S-silencer lines (S21, S26), AS-silencer lines (AS4, AS10), and double-strand RNA (IR)-silencer line (IR8) compared to wild type Col-0 control (FIGS. 4a-4e, FIGS. 11a-11f. For DNA methylation analysis at the silencer region, AS-silencer transgenic line AS27, which did not produce any sRNAs nor show any tmm-phenotype (data not shown), was used as a control. FIG. 4e shows significant increase of CG and CHG methylation in AS-silencer lines with sRNAs and tmm-phenotype (FIG. 4e, FIGS. 12a-12c). A slight increase of CHH methylation was observed which was different from that of the target region (FIGS. 4d, 4e, 11f, 4c).

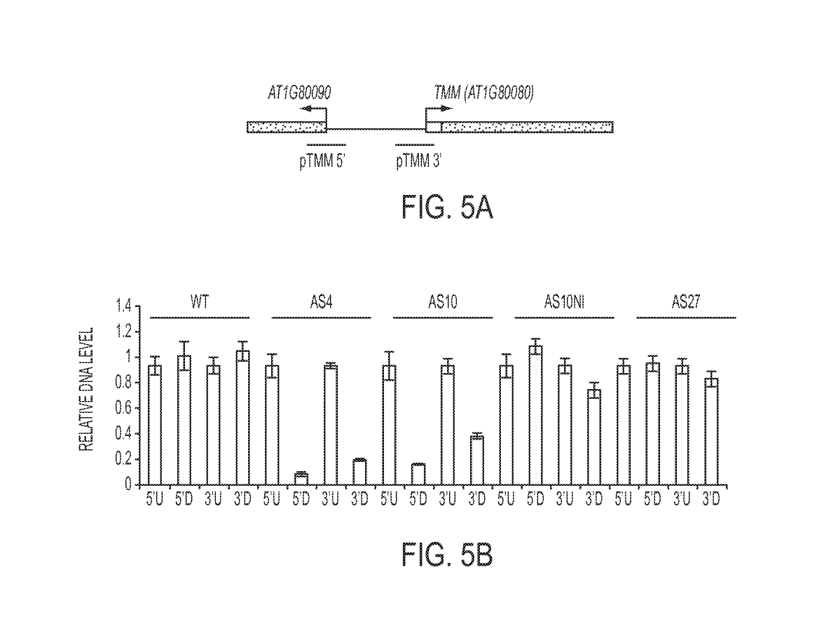

To determine whether the DNA methylation at TMM promoter region was silencer-sRNAs dependent, we used McrBc digestion analysis as a rapid assay. There was no significant DNA methylation in the AS10NI line, which was segregated from the heterozygous AS10 parent and carried no T-DNA insert nor silencer (FIGS. 5a and 5b). Therefore, these data suggest that the DNA methylation at the TMM promoter region may require the presence of silencer from which the sRNAs can be produced.

### Example 6

### Histone Modifications Observed at the TMM Promoter Region

Modifications at specific positions of histone on nucleosomes play an important roles to shape the chromatin structure and to regulate transcription activity in plants (Loidl, 2004). Two groups of histone modifications have been identified: 1) active or euchromatin markers associated with regions of actively expressed genes; 2) repressive or heterochromatin markers associated with inactive regions in euchromotin or heterochromotin (Loidl, 2004; Bernatavichute et al., 2008; Liu et al., 2010). In rice, DNA methylation of targeted endogenous promoters was for the most part not linked to TGS nor formation of repressive chromatin (Okano et al., 2008).

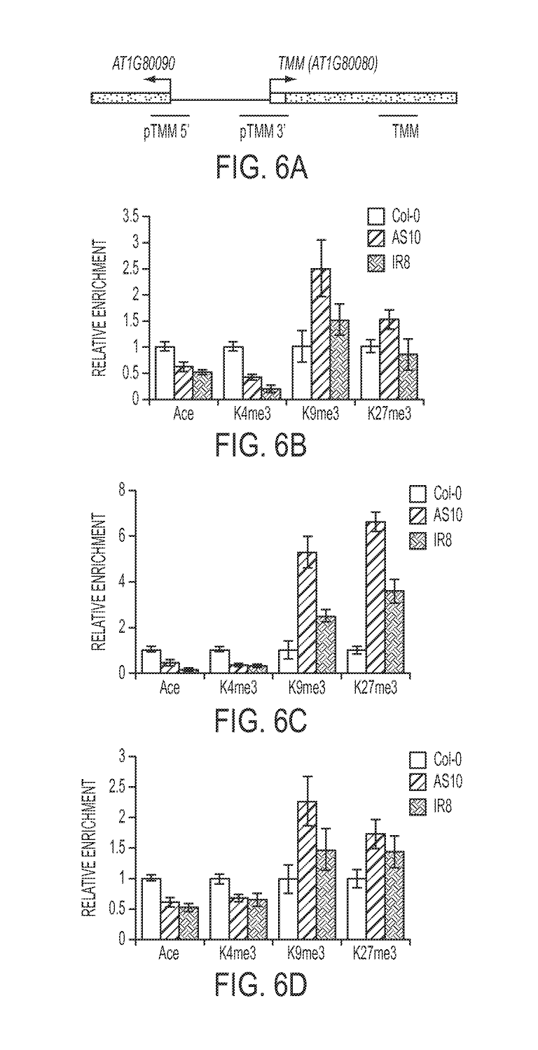

To determine whether the changes in DNA methylation observed at the TMM promoter region were accompanied by changes in histone modifications, we used chromatin immunoprecipitation (ChIP) assay to investigate H3 acetylation, H3K4 trimethylation for active markers and H3K9me3 and H3K27me3 for repressive markers. Three primer pairs were designed to examine both promoter (5′ and 3′) and coding region of the endogenous TMM locus (FIG. 6a). Significant reduction of active markers, H3K9/14Ac and H3K4me3 was observed in AS-silencer and IR-silencer transgenic plants at promoter regions, compared to untransformed WT plants (FIGS. 6b and 6c). Accordingly, high levels of H3K9me3 and H3K27me3 were seen at the 3′ region of the TMM promoter while such enrichment of these repressive markers was less significant at the 5′ promoter and coding regions (FIGS. 6b, 6c and 6d). Overall, both AS-silencer and IR-silencer plants possessed similar patterns of histone modifications which were correlated with DNA methylation at the TMM promoter region mediated by sRNAs.

### Example 7

### Single-Stranded Silencers Targeting FHY1, HFR1, and PhyB Promoters can Induce TGS of Target Gene

FHY1 (far-red elongated hypocotyl) and HFR1 (long hypocotyl in far red) encode proteins involved in positive regulation of phytochrome A signaling pathway. Under far-red light condition, elongated hypocotyls were observed in seedlings of fhy1 and hfr1 mutants (Whitelam et al., 1993; Fairchild et al., 2000; Soh et al., 2000). PhyB is one of the five phytochromes in Arabidopsis and phyB null mutants display longer hypocotyls compared to WT under red light (Reed et al., 1993). Similar to the TMM-silencers, we constructed S- and AS-silencer vectors targeting promoters of FHY1, HFR1, and PhyB and generated transgenic plants for phenotypic observations. For FHY1, an IR-silencer was also made and the corresponding transgenic plants were harvested simultaneously.

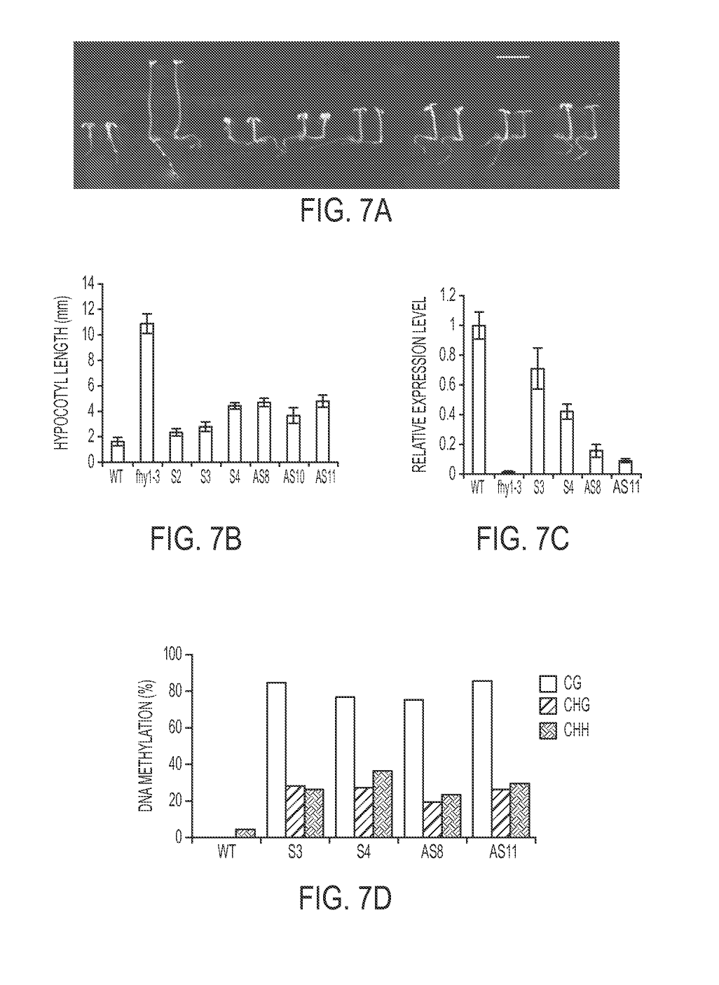

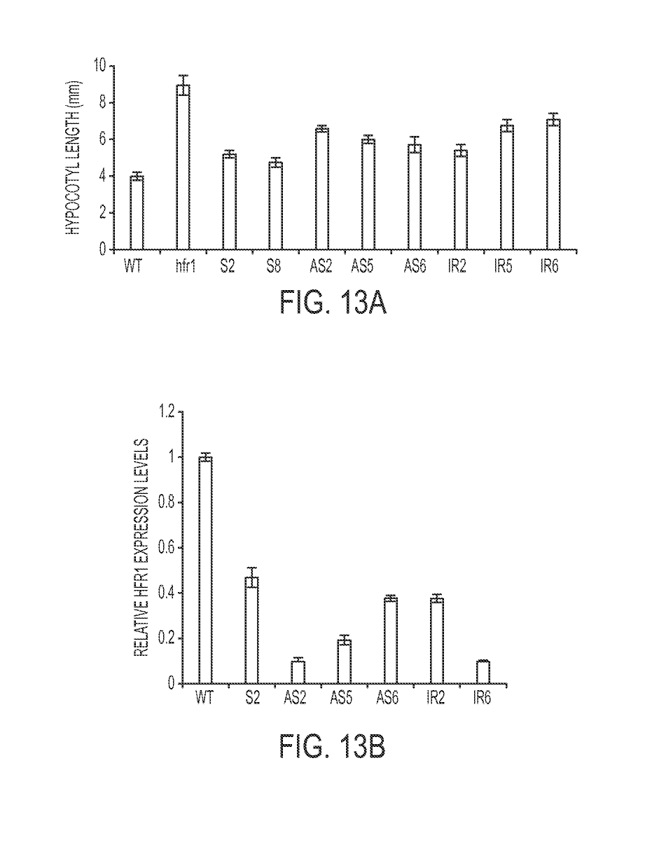

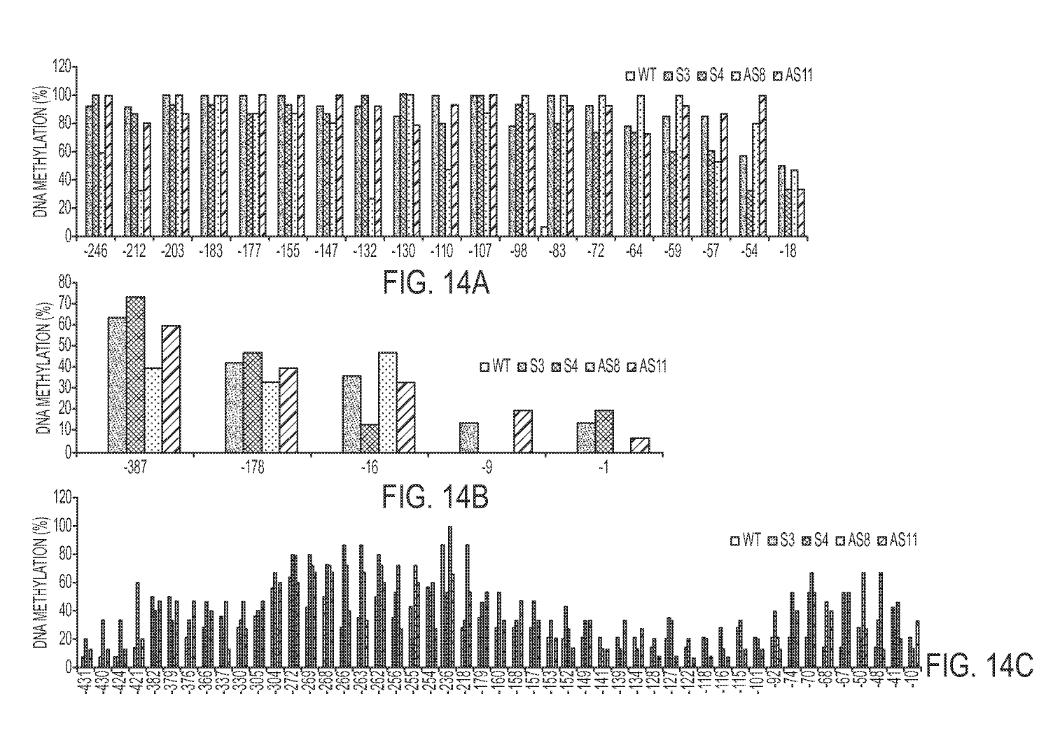

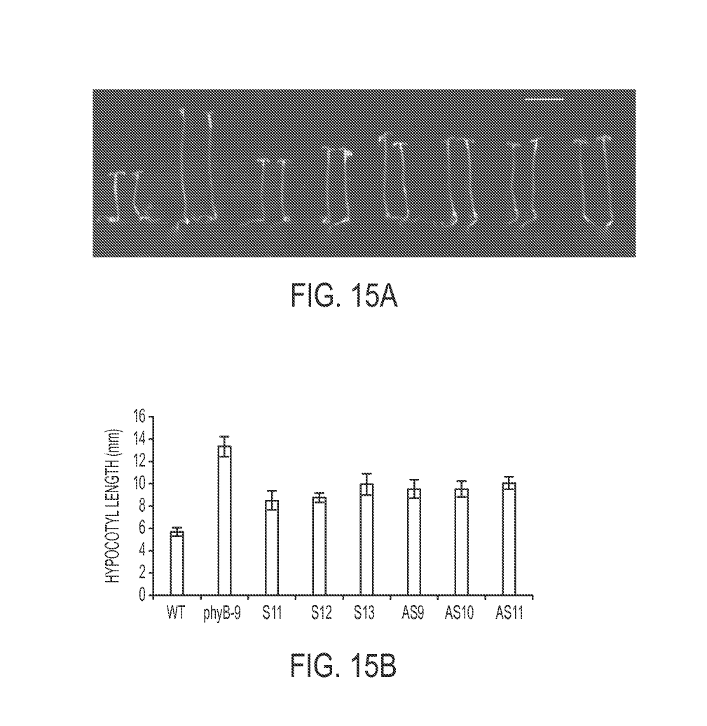

FIGS. 7a and 7b and FIG. 13a show hypocotyl lengths of in transgenic lines bearing either S-silencer or AS-silencer targeting FHY1 or HFR1. The increase in hypocotyl length was significant but the phenotype was moderate compared to null mutants of fhy1-3 and hfr1-201. Quantitative RT-PCR results confirmed the reduction of FHY1 and HFR1 transcripts in the transgenic lines (FIG. 7c, FIG. 13b). Sequencing of bisulfite-treated DNA revealed significantly increase of DNA methylation at the endogenous FHY1 promoter in both the S-silencer (S3, S4) and the AS-silencer (AS8, AS11) transgenic plants (FIG. 7d, FIGS. 14a-14c). Elongated hypocotyls of transgenic plants carrying S-silencer and AS-silencer targeting PhyB promoter under red light confirmed this strategy for TGS also worked for the PhyB locus (FIGS. 15a and 15b).

### Example 8

### Discussion

We have demonstrated that by using conventional IR silencing, two Arabidopsis endogenes, TMM and HFR1, can be efficiently silenced. (FIG. 1, FIG. 13). This is different from the results reported for rice, in which only 1 out of 7 endogenes could be moderately silenced by IR silencer. Furthermore, using single-stranded silencers targeting on promoter regions we were able to silence 4 Arabidopsis endogenes: TMM, FHY1, HFR1, and PhyB. Results indicate that single-stranded AS silencer has comparable efficiency to double-stranded IR silencer, whereas single-stranded S silencer has a much weaker effect (FIGS. 1a-1d, Table 2). This result is in contrast to a previous report that single-stranded silencers cannot trigger efficient TGS as inverted repeat silencer using a transgenic system (Mette et al., 2000). Since Mette et al. (2000) used a transgene as a target the difference could be due to a difference in TGS between endogenes and transgenes.

Mette et al. (2000) previously noted a good correlation between sRNAs and DNA methylation in TGS of transgenes. Double-stranded IR silencer can produce ˜24nt sRNAs efficiently and trigger DNA methylation and TGS of homologous sequences in trans. However, neither DNA methylation nor TGS was observed with single-stranded S- or AS-silencer alone or even by transcribed S- and AS-silencers together in transgenic plants (Mette et al., 2000). Other than tobacco and Arabidopsis, TGS initiated by sRNAs targeting on promoter was also observed in petunia (Sijen et al., 2001) and maize (Cigan et al., 2005). However, promoter-targeted sRNAs did not trigger TGS of endogenes in the monocot rice even though promoter DNA methylation was observed (Okano et al., 2008). This result indicates that sRNA-mediated DNA methylation alone is not sufficient to induce TGS, at least in some cases. Here, we found sRNAs targeted to the TMM promoter are necessary for DNA methylation and gene silencing. In WT, no sRNAs associated with TMM locus were recovered by sRNA sequencing (FIG. 3a, Table 5). Consistent with this, no promoter DNA methylation was detected (FIG. 4) and the promoter remains active. All tested silenced transgenic lines produced promoter-related sRNAs and displayed promoter DNA methylation (FIG. 3a, 4). Transgenic lines such as AS27 without promoter-related sRNAs also did not show any DNA methylation of the TMM promoter (FIG. 5). Moreover, in silenced lines the tmm mutant phenotype reverted to WT when the transgenic silencer was segregated by genetic crosses, e.g. AS10NI and IR8NI (FIG. 1), and no TMM promoter-related sRNAs were detected in progeny plants not carrying the silencer transgene (FIG. 3a, Table 5).

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

Agorio, A., and Vera, P. (2007). ARGONAUTE4 is required for resistance to Pseudomonas syringae in Arabidopsis. The Plant Cell 19, 3778-3790.
Aufsatz, W., Mette, M. F., Van Der Winden, J., Matzke, A. J. M., and Matzke, M. (2002). RNA-directed DNA methylation in Arabidopsis. Proceedings of the National Academy of Sciences of the United States of America 99, 16499-16506.
Bernatavichute, Y. V., Zhang, X., Cokus, S., Pellegrini, M., and Jacobsen, S. E. (2008). Genome-wide association of histone H3 lysine nine methylation with CHG DNA methylation in Arabidopsis thaliana. PLoS One 3, e3156.
Chiu, W. et al. (1996). Engineered GFP as a vital reporter in plants. Current Biology 6:325-330.
Christensen, A. H. and Quail, P. H, (1989). Sequence analysis and transcriptional regulation by heat shock of polyubiquitin transcripts from maize. Plant Mol Biol 12:619-632.
Christensen, A. H. et al. (1992). Maize polyubiquitin genes: structure, thermal perturbation of expression and transcript splicing, and promoter activity following transfer to protoplasts by electroporation. Plant Mol Biol 18:675-689.
Cigan, A. M., Unger-Wallace, E., and Haug-Collet, K. (2005). Transcriptional gene silencing as a tool for uncovering gene function in maize. The Plant Journal 43, 929-940.
Daxinger, L., Kanno, T., Bucher, E., Van Der Winden, J., Naumann, U., Matzke, A. J. M., and Matzke, M. (2009). A stepwise pathway for biogenesis of 24-nt secondary siRNAs and spreading of DNA methylation. The EMBO Journal 28, 48-57.
De Wet, J. R. et al. (1987). Firefly luciferase gene: structure and expression in mammalian cells. Mol Cell Biol 7:725-737.
Eamens, A., Wang, M. B., Smith, N. A., and Waterhouse, P. M. (2008). RNA silencing in plants: yesterday, today, and tomorrow. Plant Physiology 147, 456-468.
Elmayan, T., Balzergue, S., Béon, F., Bourdon, V., Daubremet, J., Guénet, Y., Mourrain, P., Palauqui, J. C., Vernhettes, S., and Vialle, T. (1998). Arabidopsis mutants impaired in cosuppression. The Plant Cell 10, 1747-1758.
Fairchild, C. D., Schumaker, M. A., and Quail, P. H. (2000). HFR1 encodes an atypical bHLH protein that acts in phytochrome A signal transduction. Genes & Development 14, 2377-2391.
Garcia-Ruiz, H., Takeda, A., Chapman, E. J., Sullivan, C. M., Fahlgren, N., Brempelis, K. J., and Carrington, J. C. (2010). Arabidopsis RNA-dependent RNA polymerases and dicer-like proteins in antiviral defense and small interfering RNA biogenesis during Turnip Mosaic Virus infection. The Plant Cell 22, 481-496.
Goff, S. A. et al. (1990). Transactivation of anthocyanin biosynthetic genes following transfer of B regulatory genes into maize tissues. EMBO J 9:2517-2522.
Guo, H. S., Fei, J. F., Xie, Q., and Chua, N. H. (2003). A chemical-regulated inducible RNAi system in plants. The Plant Journal 34, 383-392.
Haag, J. R., and Pikaard, C. S. (2011). Multisubunit RNA polymerases IV and V: purveyors of non-coding RNA for plant gene silencing. Nature Reviews Molecular Cell Biology 12, 483-492.
Hamilton, A., Voinnet, O., Chappell, L., and Baulcombe, D. (2002). Two classes of short interfering RNA in RNA silencing. The EMBO Journal 21, 4671-4679.
Havecker, E. R., Wallbridge, L. M., Hardcastle, T. J., Bush, M. S., Kelly, K. A., Dunn, R. M., Schwach, F., Doonan, J. H., and Baulcombe, D. C. (2010). The Arabidopsis RNA-directed DNA methylation argonautes functionally diverge based on their expression and interaction with target loci. The Plant Cell 22, 321-334.
Heilersig, B. H. J. B., Loonen, A. E. H. M., Janssen, E. M., Wolters, A. M. A., and Visser, R. G. F. (2006). Efficiency of transcriptional gene silencing of GBSSI in potato depends on the promoter region that is used in an inverted repeat. Molecular Genetics and Genomics 275, 437-449.
Henderson, I. R., and Jacobsen, S. E. (2008). Tandem repeats upstream of the Arabidopsis endogene SDC recruit non-CG DNA methylation and initiate siRNA spreading. Genes & Development 22, 1597.
Henderson, I. R., Zhang, X., Lu, C., Johnson, L., Meyers, B. C., Green, P. J., and Jacobsen, S. E. (2006). Dissecting Arabidopsis thaliana DICER function in small RNA processing, gene silencing and DNA methylation patterning. Nature Genetics 38, 721-725.
Herr, A., Jensen, M., Dalmay, T., and Baulcombe, D. (2005). RNA polymerase IV directs silencing of endogenous DNA. Science 308, 118-120.
Jefferson, R. A. et al. (1991). Plant Molecular Biology Manual, ed. Gelvin et al., Kluwer Academic Publishers, pp. 1-33.
Jones, L., Ratcliff, F., and Baulcombe, D. C. (2001). RNA-directed transcriptional gene silencing in plants can be inherited independently of the RNA trigger and requires Met1 for maintenance. Current Biology 11, 747-757.
Kain, S. R. et al. (1995). Green fluorescent protein as a reporter of gene expression and protein localization. BioTechniques 19, 650-655.
Kanno, T., Mette, M. F., Kreil, D. P., Aufsatz, W., Matzke, M., and Matzke, A. J. M. (2004). Involvement of putative SNF2 chromatin remodeling protein DRD1 in RNA-directed DNA methylation. Current Biology 14, 801-805.
Kanno, T., Huettel, B., Mette, M. F., Aufsatz, W., Jaligot, E., Daxinger, L., Kreil, D. P., Matzke, M., and Matzke, A. J. M. (2005). Atypical RNA polymerase subunits required for RNA-directed DNA methylation. Nature Genetics 37, 761-765.
Kost, B., Spielhofer, P., and Chua, N. H. (1998). A GFP-mouse talin fusion protein labels plant actin filamentsin vivoand visualizes the actin cytoskeleton in growing pollen tubes. The Plant Journal 16, 393-401.
Last, D. I. et al. (1991). pEmu: an improved promoter for gene expression in cereal cells. Theor Appl Genet 81:581-588.
Law, J. A., and Jacobsen, S. E. (2010). Establishing, maintaining and modifying DNA methylation patterns in plants and animals. Nature Reviews Genetics 11, 204-220.
Liu, C., Lu, F., Cui, X., and Cao, X. (2010). Histone methylation in higher plants. Annual Review of Plant Biology 61, 395-420.
Liu, Y. G., Mitsukawa, N., Oosumi, T., and Whittier, R. F. (1995). Efficient isolation and mapping of Arabidopsis thaliana T-DNA insert junctions by thermal asymmetric interlaced PCR. The Plant Journal 8, 457-463.
Loidl, P. (2004). A plant dialect of the histone language. Trends in Plant Science 9, 84-90.
Mallory, A., and Vaucheret, H. (2010). Form, function, and regulation of ARGONAUTE proteins. The Plant Cell 22, 3879-3889.
Matzke, M., Primig, M., Trnovsky, J., and Matzke, A. (1989). Reversible methylation and inactivation of marker genes in sequentially transformed tobacco plants. The EMBO Journal 8, 643-649.
Matzke, M., Kanno, T., Daxinger, L., Huettel, B., and Matzke, A. J. M. (2009). RNA-mediated chromatin-based silencing in plants. Current Opinion in Cell Biology 21, 367-376.
Matzke, M. A., and Matzke, A. J. M. (2004). Planting the seeds of a new paradigm. PLoS Biology 2, e133.
McElroy, D. et al. (1990). Isolation of an efficient actin promoter for use in rice transformation. Plant Cell 2:163-171.
Mette, M., Aufsatz, W., Van der Winden, J., Matzke, M., and Matzke, A. (2000). Transcriptional silencing and promoter methylation triggered by double-stranded RNA. The EMBO Journal 19, 5194-5201.
Miura, A., Nakamura, M., Inagaki, S., Kobayashi, A., Saze, H., and Kakutani, T. (2009). An Arabidopsis jmjC domain protein protects transcribed genes from DNA methylation at CHG sites. The EMBO Journal 28, 1078-1086.
Mlotshwa, S., Pruss, G. J., Gao, Z., Mgutshini, N. L., Li, J., Chen, X., Bowman, L. H., and Vance, V. (2010). Transcriptional silencing induced by Arabidopsis T-DNA mutants is associated with 35S promoter siRNAs and requires genes involved in siRNA-mediated chromatin silencing. The Plant Journal 64, 699-704.
Odell, J. T. et al. (1985). Identification of DNA sequences required for activity of the cauliflower mosaic virus 35S promoter. Nature 313:810-812.
Okano, Y., Miki, D., and Shimamoto, K. (2008). Small interfering RNA (siRNA) targeting of endogenous promoters induces DNA methylation, but not necessarily gene silencing, in rice. The Plant Journal 53, 65-77.
Pontier, D., Yahubyan, G., Vega, D., Bulski, A., Saez-Vasquez, J., Hakimi, M. A., Lerbs-Mache, S., Colot, V., and Lagrange, T. (2005). Reinforcement of silencing at transposons and highly repeated sequences requires the concerted action of two distinct RNA polymerases IV in Arabidopsis. Genes & Development 19, 2030-2040.
Reed, J. W., Nagpal, P., Poole, D. S., Furuya, M., and Chory, J. (1993). Mutations in the gene for the red/far-red light receptor phytochrome B alter cell elongation and physiological responses throughout Arabidopsis development. The Plant Cell 5, 147-157.
Sijen, T., Vijn, I., Rebocho, A., van Blokland, R., Roelofs, D., Mol, J. N. M., and Kooter, J. M. (2001). Transcriptional and posttranscriptional gene silencing are mechanistically related. Current Biology 11, 436-440.
Soh, M. S., Kim, Y. M., Han, S. J., and Song, P. S. (2000). REP1, a basic helix-loop-helix protein, is required for a branch pathway of phytochrome A signaling in Arabidopsis. The Plant Cell 12, 2061-2074.
Vaucheret, H. (2006). Post-transcriptional small RNA pathways in plants: mechanisms and regulations. Genes & Development 20, 759-771.
Velten, J. et al. (1984). Isolation of a dual plant promoter fragment from the Ti plasmid of Agrobacterium tumefaciens. EMBO J3, 2723-2730.
Wassenegger, M., Heimes, S., Riedel, L., and Sänger, H. L. (1994). RNA-directed de novo methylation of genomic sequences in plants. Cell 76, 567-576.
Whitelam, G. C., Johnson, E., Peng, J., Carol, P., Anderson, M. L., Cowl, J. S., and Harberd, N. P. (1993). Phytochrome A null mutants of Arabidopsis display a wild-type phenotype in white light. The Plant Cell 5, 757-768.
Willmann, M. R., Endres, M. W., Cook, R. T., and Gregory, B. D. (2011). The Functions of RNA-Dependent RNA Polymerases in Arabidopsis. The Arabidopsis Book 9, e0146.
Wu, H. W., Lin, S. S., Chen, K. C., Yeh, S. D., and Chua, N. H. (2010). Discriminating mutations of HC-Pro of Zucchini yellow mosaic virus with differential effects on small RNA pathways involved in viral pathogenicity and symptom development. Molecular Plant-Microbe Interactions 23, 17-28.
Xie, Z., Johansen, L. K., Gustafson, A. M., Kasschau, K. D., Lellis, A. D., Zilberman, D., Jacobsen, S. E., and Carrington, J. C. (2004). Genetic and functional diversification of small RNA pathways in plants. PLoS Biology 2, e104.
Zeidler, M., Zhou, Q., Sarda, X., Yau, C. P., and Chua, N. H. (2004). The nuclear localization signal and the C-terminal region of FHY1 are required for transmission of phytochrome A signals. The Plant Journal 40, 355-365.
Zhang, X., Henriques, R., Lin, S. S., Niu, Q. W., and Chua, N. H. (2006a). Agrobacterium-mediated transformation of Arabidopsis thaliana using the floral dip method. Nature Protocols 1, 641-646.
Zhang, X., Yazaki, J., Sundaresan, A., Cokus, S., Chan, S. W. L., Chen, H., Henderson, I. R., Shinn, P., Pellegrini, M., and Jacobsen, S. E. (2006b). Genome-wide high-resolution mapping and functional analysis of DNA methylation in Arabidopsis. Cell 126, 1189-1201.
Zilberman, D., Gehring, M., Tran, R. K., Ballinger, T., and Henikoff, S. (2006). Genome-wide analysis of Arabidopsis thaliana DNA methylation uncovers an interdependence between methylation and transcription. Nature Genetics 39, 61-69.

1. A nucleic acid construct comprising a plant operable promoter operatively linked to a nucleic acid silencer molecule operatively linked to plant operable 3′ regulatory region, wherein the nucleic acid silencer molecule comprises at least one promoter region of a plant endogene target, wherein expression of the nucleic acid silencer molecule in a transgenic plant results in transcriptional gene silencing of the plant endogene target.

2. The nucleic acid construct of claim 1, wherein the at least one promoter region is a single copy of a promoter region.

3. The construct of claim 1, wherein the nucleic acid silencer molecule is in a sense orientation with respect to the plant operable promoter.

4. The construct of claim 3, wherein transcription of the nucleic acid silencer molecule produces a single-stranded sense silencer.

5. The construct of claim 1, wherein the nucleic acid silencer molecule is in an antisense orientation with respect to the plant operable promoter.

6. The construct of claim 5, wherein transcription of the nucleic acid silencer molecule produces a single-stranded antisense silencer.

7. The construct of claim 1, wherein the nucleic acid silencer molecule is provided in an inverted repeat configuration.

8. The construct of claim 7, wherein transcription of the inverted repeat nucleic acid silencer molecule produces a double-stranded silencer.

9. The construct of claim 7, wherein the nucleic acid construct additionally comprises a spacer sequence between the inverted repeat silencer regions.

10. The construct of claim 1, wherein the plant operable promoter is a single promoter, a double homologous promoter or a double heterologous promoter.

11. The construct of claim 1, wherein the 3′ regulatory region is a 3′ viral sequence.

12. The construct of claim 1, wherein the 3′ regulatory region is a polyA addition sequence.

13. The construct of claim 1, wherein the promoter region comprises about 300 contiguous nucleotides to about 1500 contiguous nucleotides of the endogene, about 400 contiguous nucleotides to about 1200 contiguous nucleotides of the endogene, about 425 contiguous nucleotides to about 1100 contiguous nucleotides of the endogene or about 425 contiguous nucleotides to about 1075 contiguous nucleotides of the endogene.

14. The construct of claim 13, wherein the promoter region comprises a nucleotide sequence selected from the group consisting of SEQ ID NO:1, SEQ ID NO:2, SEQ ID NO:3 and SEQ ID NO:4.

15. A nucleic acid construct comprising a plant operable promoter operatively linked to a nucleic acid silencer molecule operatively linked to plant operable 3′ regulatory region, wherein the nucleic acid silencer molecule comprises at least one promoter region of a plant endogene target, wherein the at least one promoter region is not present as an inverted repeat, and wherein expression of the nucleic acid silencer molecule in a transgenic plant results in transcriptional gene silencing of the plant endogene target.

16. The nucleic acid construct of claim 15, wherein the at least one promoter region is a single copy of a promoter region.

17. The construct of claim 15, wherein the promoter region of the nucleic acid silencer molecule is in a sense orientation with respect to the plant operable promoter.

18. The construct of claim 17, wherein transcription of the promoter region of the nucleic acid silencer molecule produces a single-stranded sense silencer.

19. The construct of claim 17, wherein the nucleic acid silencer molecule is in an antisense orientation with respect to the plant operable promoter.

20. The construct of claim 19, wherein transcription of the nucleic acid silencer molecule produces a single-stranded antisense silencer.

21. The construct of claim 15, wherein the plant operable promoter is a single promoter, a double homologous promoter or a double heterologous promoter.

22. The construct of claim 15, wherein the 3′ regulatory region is a 3′ viral sequence.

23. The construct of claim 15, wherein the 3′ regulatory region is a polyA addition sequence.

24. The construct of claim 15, wherein the promoter region comprises about 300 contiguous nucleotides to about 1500 contiguous nucleotides of the endogene, about 400 contiguous nucleotides to about 1200 contiguous nucleotides of the endogene, about 425 contiguous nucleotides to about 1100 contiguous nucleotides of the endogene or about 425 contiguous nucleotides to about 1075 contiguous nucleotides of the endogene.

25. The construct of claim 24, wherein the promoter region comprises a nucleotide sequence selected from the group consisting of SEQ ID NO:1, SEQ ID NO:2, SEQ ID NO:3 and SEQ ID NO:4.

26. A plant transformation vector comprising the nucleic acid construct of claim 1.

27. A transgenic plant cell comprising the nucleic acid construct of claim 1 stably incorporated into its genome.

28. A transgenic plant comprising the nucleic acid construct of claim 1 stably incorporated into its genome.

29. A method of reducing expression of a plant endogene via transcriptional gene silencing in a plant cell comprising culturing the transgenic plant cell of claim 27 under conditions suitable for expression of the nucleic acid silencer molecule, whereby expression of the plant endogene is reduced.

30. The method of claim 29, wherein expression of the nucleic acid silencer molecule results in production of an initial single-stranded transcript, followed by processing of the initial single-stranded transcript to produce small RNAs in the transgenic plant cell, followed by transcriptional gene silencing of the plant endogene by the small RNAs in the transgenic plant cell,

31. A method of reducing expression of a plant endogene via transcriptional gene silencing in a plant comprising growing the transgenic plant of claim 28 under conditions suitable for expression of the nucleic acid silencer molecule, whereby expression of the plant endogene is reduced.

32. The method of claim 31, wherein expression of the nucleic acid silencer molecule leads to production of an initial single-stranded transcript, followed by processing of the initial single-stranded transcript to produce small RNAs in the transgenic plant, followed by transcriptional gene silencing of the plant endogene in the transgenic plant by the small RNAs.

33. A method of reducing expression of a plant endogene via transcriptional gene silencing in a plant cell comprising
transforming a plant cell with the nucleic acid construct of claim 1 to produce a transgenic plant cell having the nucleic acid construct or the plant transformation vector stably integrated in its genome and
culturing the transgenic plant cell under conditions suitable for expression of the nucleic acid silencer molecule, whereby expression of the plant endogene is reduced.

34. The method of claim 33, wherein expression of the nucleic acid silencer molecule leads to production of an initial single-stranded transcript, followed by processing of the initial single-stranded transcript to produce small RNAs in the transgenic plant cell, followed by transcriptional gene silencing of the plant endogene by the small RNAs in the transgenic plant cell.

35. A method of reducing expression of a plant endogene via transcriptional gene silencing in a plant comprising
transforming a plant cell with the nucleic acid construct of claim 1 to produce a transgenic plant cell having the nucleic acid construct or the plant transformation vector stably integrated in its genome,
regenerating a transgenic plant from the transgenic plant cell, wherein the transgenic plant has the nucleic acid construct or the plant transformation vector stably integrated in its genome and
growing the transgenic plant under conditions suitable for expression of the nucleic acid silencer molecule, whereby expression of the plant endogene is reduced.

36. The method of claim 35, wherein expression of the nucleic acid silencer molecule leads to production of an initial single-stranded transcript, followed by processing of the initial single-stranded transcript to produce small RNAs in the transgenic plant, followed by transcriptional gene silencing of the plant endogene in the transgenic plant by the small RNAs.

37. A method to identify a fragment of a plant promoter useful for transcriptional gene silencing of a plant endogene, the method comprising
(a) transforming a plant cell with the construct of claim 1 and
(b) testing for transcriptional gene silencing in a transformed plant cell.

38. The method of claim 37, wherein the testing comprises the use of one or more of the following methods: RT-PCR, PCR, northern blot, immunological assay or enzymatic assay.

39. The method of claim 38, wherein the testing comprises the use of an immunological assay.

40. The method of claim 39, wherein the immunological assay comprises the use of antibodies directed to sites of histone acetylation or histone methylation.

41. The method of claim 38, wherein the testing comprises the use of an enzymatic assay.

42. The method of claim 41, wherein the enzymatic assay comprises digesting DNA with McrBc.

43. The method of claim 37, which further comprises
(a1) culturing the transformed plant cell under conditions suitable for expression of the putative nucleic acid silencer molecule.

44. The method of claim 37, which further comprises
(a1) regenerating a transformed plant from the transformed plant cell and
(a2) growing the transformed plant under conditions suitable for expression of the putative nucleic acid silencer molecule in transformed plant cells.

45. The method of claim 37, which further comprises identifying the putative nucleic acid silencing molecule as a nucleic acid silencing molecule of a target plant endogene if there has been transcriptional gene silencing in a transformed plant cell.

46. A method of reducing expression of a plant endogene via transcriptional gene silencing in a plant cell comprising
transforming a plant cell with the plant transformation vector of claim 26 to produce a transgenic plant cell having the nucleic acid construct or the plant transformation vector stably integrated in its genome and
culturing the transgenic plant cell under conditions suitable for expression of the nucleic acid silencer molecule, whereby expression of the plant endogene is reduced.

47. A method of reducing expression of a plant endogene via transcriptional gene silencing in a plant comprising
transforming a plant cell with the plant transformation vector of claim 26 to produce a transgenic plant cell having the nucleic acid construct or the plant transformation vector stably integrated in its genome,
regenerating a transgenic plant from the transgenic plant cell, wherein the transgenic plant has the nucleic acid construct or the plant transformation vector stably integrated in its genome and
growing the transgenic plant under conditions suitable for expression of the nucleic acid silencer molecule, whereby expression of the plant endogene is reduced.

48. A method to identify a fragment of a plant promoter useful for transcriptional gene silencing of a plant endogene, the method comprising
(a) transforming a plant cell with the plant transformation vector of claim 26 and
(b) testing for transcriptional gene silencing in a transformed plant cell.

49. The method of claim 48, wherein the testing comprises the use of one or more of the following methods: RT-PCR, PCR, northern blot, immunological assay or enzymatic assay.

50. The method of claim 48, which further comprises
(a1) culturing the transformed plant cell under conditions suitable for expression of the putative nucleic acid silencer molecule.

51. The method of claim 48, which further comprises
(a1) regenerating a transformed plant from the transformed plant cell and
(a2) growing the transformed plant under conditions suitable for expression of the putative nucleic acid silencer molecule in transformed plant cells.

52. The method of claim 37, which further comprises identifying the putative nucleic acid silencing molecule as a nucleic acid silencing molecule of a target plant endogene if there has been transcriptional gene silencing in a transformed plant cell.


SEQUENCE LISTING (55 sequences)

SEQ ID NO: 1 - DNA, 475 bp, Arabidopsis thaliana
tgttgctccatgggcatgtgcttttcgtatgcacagaccacgcgtccgtttattcagattctcgtgttacaacaaaataatgattttaaggttcagaatcaaacatatgaaactttttatcaattgctatctacggtttgttacaaaaacaaatacagtgcccagttcaaaatacaaattgaataatagcccaacatatcgtaaatgggaaaatgggctcagcccaaataaaaaaaaagtaaacgataaggagacgtcgggtcccgttcacgaagcggtcggatctagggcccaacacaagacgaatgaagggaatatctcgtgaaagcaataaatgcagaaatcgagaatcatgaattttatttatttcgctcctcgagttatcccgtgaaagtcataaagacattgcgaacgtttttatggacccaccaattgtttcttcagaattatacatatacacactgacaatttcgaa

SEQ ID NO: 2 - DNA, 449 bp, Arabidopsis thaliana
aatttttgtaaaaaaaatccaaattcttctaaaattaagtttttaatagaaatttaaaaagacagttctacatcatattattttctttaaataatgatataaaatttataaactattatctttgtaagataatgaatatttatacctatgtaagggaaaaattgataaatatttatacatcctcatccaaaagcccataaaatcgttatgggacaatagtaaaaaagattgcaaaatcgtatggagcgaaagataaaaataaaaaacggtcccgtgtaagaagatgggtcacctcgccaacacgtagactcttttcacgcgccaaatcaaacaccatgtcgacgagatcaacggattcaaatgtttcgtgtaatacacgctccaacgtagcgcgtcgtacacttgatgcttaatttatgtattttaagttacgcagtttccagattttc

SEQ ID NO: 3 - DNA, 1025 bp, Arabidopsis thaliana
ttggataaaaccctaaatgaattcttttttcaggaatccataaatggtcatgaaatgaaatgcattctcaggctggaaataatcgaccattatattattgcacaaaaaagtgaactctagtatcataactctgaaattattatgattgctggttactgaattttgtaagcctttcataataacgaatttaccaattgggagatcgaataatcttatcgttgttacagacgatatttttttgtcgattatattatgaaacttgtggtagtcaacctttttttttccaaacggagacgtcatctttttgtttttatatttagagatgatctgaccaaaaataaacaaatagacatccgatattttacattgttttatatatatataaagataaagcttaatgacgagtattcaatgataaacgataagattacgggaaaaataattaaacggataaaaatggccctacccgttgtgcattacagttcttggttaagaaatactattaaactgtctctcaaatctcaatatataaatgaatacattctatacgtacaaagataaatttatctctatgttcatgtgtcaaagtggtagtgaatctagtgacgttattgattaatcaagattacttttatctttctgtgcaaaatttcaagattactctaaagtcgatatatgctactatgacgtagttttgggacaattacgtatagaatcataaaaacaataagcagaataataatccatttaagtacattgtgaatttgtgataatggaattatttgacaatacttttccaaaataaaaactaatttaattactgtattaattattttagtaaaacaatattttcattacaatgtttgttgtgaatccaaaaaagtattgtttccaaagaactcaaatgacatcaatatgtctataataacctcgtgtagctgtatgttagaatctagccgaattgttttattgttgtcggtgtacgcaacaaacgaaccacacatactttgaaacgtggcacaata

SEQ ID NO: 4 - DNA, 1067 bp, Arabidopsis thaliana
aaattagtcattcaacaaaagaaaaggacgaacgcagtgacttaacatgaaactgtgagcggcccaaaatcatttatgtaatggacccttaacttttcatgcacacgatttttctcatttatatgtttttctgctctctttttttcctctttatcattactttaatttattttatgttcttttttcgaagcaccataattgtatgctttcaccaaataatccaaatttagaatcattaatatgtcaaaaaagaattgcatatattcaataaaacgtaatgctaagtagtacaatgcatgtattatacaaaatgtaatgatatagatccaacgtatatatcaaagtggaccaaaatatatcttatgtattagacgagtttactatgcaaaatttatgattctattccgcatggagcgtgctaatactacttcgaacccctttgagaccaatatgtgattctatattctatctagtacaaattatgagaagtatatacgtacgatgagagtataaaacatttcaatatttgtatagagaggacaccacttggttgacttgacccacgataagatattgaagaaaccaaacttgtatagtacgaattcgaaatcgtaattgatgatgcgattcgacaagtccaggggctccctcccacgcgcaatgggcccagcaaccacgtgtggccactagagagaataaaccattagcccacgtgatcttgggcccaatcaatctctccctcacattaaacgacaaaacaaaagctcttctgggttaaattgataaatatcaaaactttaaaggtaatttgctaaaatcgccacacaaaaaaagtcgcagaaaatatatgaggaaacaaaaagcgaagacgacaaaaaaaaaaaaaactctgatttttttttgttatctctctctatctgagaggcacacattttgcttcgtcttcttcaatttattttattggtttctccacttatctccgatctcaattctccccattttcttcttcctcaagttcaaaattcttgagaatttagctctaccagaattcgtctccgataactagt

SEQ ID NO: 5 - DNA, 34 bp, Arabidopsis thaliana
gggagctcgtaatcaggatcctaggtgtttcctg

SEQ ID NO: 6 - DNA, 25 bp, Arabidopsis thaliana
caaaccgcctctccccgggcgttgg

SEQ ID NO: 7 - DNA, 26 bp, Arabidopsis thaliana
cgcgtctggatccccgggcatgtccc

SEQ ID NO: 8 - DNA, 23 bp, Arabidopsis thaliana
gtgcctagggttctcccgtttcg

SEQ ID NO: 9 - DNA, 27 bp, Arabidopsis thaliana
ccgctcatgatcttgatcccctgcgcc

SEQ ID NO: 10 - DNA, 26 bp, Arabidopsis thaliana
gtaaaaatactctagaccggcctctc

SEQ ID NO: 11 - DNA, 32 bp, Arabidopsis thaliana
gacacgtattagatctgttgctccatgggcat

SEQ ID NO: 12 - DNA, 26 bp, Arabidopsis thaliana
ggactagtttcgaaattgtcagtgtg

SEQ ID NO: 13 - DNA, 25 bp, Arabidopsis thaliana
gttcctaggtgttgctccatgggca

SEQ ID NO: 14 - DNA, 27 bp, Arabidopsis thaliana
gaatgagatcttcgaaattgtcagtgt

SEQ ID NO: 15 - DNA, 20 bp, Arabidopsis thaliana
aaaataatgattttaaggtt

SEQ ID NO: 16 - DNA, 20 bp, Arabidopsis thaliana
aattcatatcrtrccatttc

SEQ ID NO: 17 - DNA, 23 bp, Arabidopsis thaliana
aagyaagtggattgatgtgatat

SEQ ID NO: 18 - DNA, 24 bp, Arabidopsis thaliana
cctaaaacttcaracacrratcta

SEQ ID NO: 19 - DNA, 23 bp, Arabidopsis thaliana
gtggaggatgaggagaaagctga

SEQ ID NO: 20 - DNA, 24 bp, Arabidopsis thaliana
ataggtgctggaccgtaccggaag

SEQ ID NO: 21 - DNA, 23 bp, Arabidopsis thaliana
ctaatctttgttttgatgctttc

SEQ ID NO: 22 - DNA, 24 bp, Arabidopsis thaliana
ctgtatttgtttttgtaacaaacc

SEQ ID NO: 23 - DNA, 20 bp, Arabidopsis thaliana
gagttatcccgtgaaagtca

SEQ ID NO: 24 - DNA, 22 bp, Arabidopsis thaliana
gaatatttggcggaagaattca

SEQ ID NO: 25 - DNA, 22 bp, Arabidopsis thaliana
catcagacggctgtacgactgc

SEQ ID NO: 26 - DNA, 24 bp, Arabidopsis thaliana
ctgtatttgtttttgtaacaaacc

SEQ ID NO: 27 - DNA, 21 bp, Arabidopsis thaliana
ctcgagttatcccgtgaaagt

SEQ ID NO: 28 - DNA, 24 bp, Arabidopsis thaliana
cggaggtgatcacagtgaaagatc

SEQ ID NO: 29 - DNA, 42 bp, Arabidopsis thaliana
taatacgactcactataggggtaagatgacacgtattacatg

SEQ ID NO: 30 - DNA, 20 bp, Arabidopsis thaliana
ttatttgggctgagcccatt

SEQ ID NO: 31 - DNA, 42 bp, Arabidopsis thaliana
taatacgactcactatagggaaaaaaagtaaacgataaggag

SEQ ID NO: 32 - DNA, 24 bp, Arabidopsis thaliana
gtgtgaatgcggtttaggttcgaa

SEQ ID NO: 33 - DNA, 40 bp, Arabidopsis thaliana
taatacgactcactatagggggtttaggttcgaaattgtc

SEQ ID NO: 34 - DNA, 22 bp, Arabidopsis thaliana
aaaaaaagtaaacgataaggag

SEQ ID NO: 35 - DNA, 40 bp, Arabidopsis thaliana
taatacgactcactatagggttatttgggctgagcccatt

SEQ ID NO: 36 - DNA, 22 bp, Arabidopsis thaliana
gtaagatgacacgtattacatg

SEQ ID NO: 37 - DNA, 38 bp, Arabidopsis thaliana
ggtctcgagatctttggataaaaccctaaatgaattct

SEQ ID NO: 38 - DNA, 35 bp, Arabidopsis thaliana
catggatcccactagtattgtgccacgtttcaaag

SEQ ID NO: 39 - DNA, 31 bp, Arabidopsis thaliana
cgtatccaggtcttaagtagtgatgatgaat

SEQ ID NO: 40 - DNA, 30 bp, Arabidopsis thaliana
cttgtgacaattaggtacgagttgctgtag

SEQ ID NO: 41 - DNA, 32 bp, Arabidopsis thaliana
agcctcgagaatttttgtaaaaaaaatccaaa

SEQ ID NO: 42 - DNA, 32 bp, Arabidopsis thaliana
agcggatccgaaaatctggaaactgcgtaact

SEQ ID NO: 43 - DNA, 32 bp, Arabidopsis thaliana
agcctcgaggaaaatctggaaactgcgtaact

SEQ ID NO: 44 - DNA, 32 bp, Arabidopsis thaliana
agcggatccaatttttgtaaaaaaaatccaaa

SEQ ID NO: 45 - DNA, 25 bp, Arabidopsis thaliana
caaattrtccaaaaaaataattacc

SEQ ID NO: 46 - DNA, 24 bp, Arabidopsis thaliana
gagagttyaaagataattgttttt

SEQ ID NO: 47 - DNA, 26 bp, Arabidopsis thaliana
tggaagatggactgttaaccaagatg

SEQ ID NO: 48 - DNA, 27 bp, Arabidopsis thaliana
tgagtctcaacatcattttgttacagc

SEQ ID NO: 49 - DNA, 31 bp, Arabidopsis thaliana
ggtctcgagaaattagtcattcaacaaaaga

SEQ ID NO: 50 - DNA, 31 bp, Arabidopsis thaliana
gtagagctcactagttatcggagacgaattc

SEQ ID NO: 51 - DNA, 34 bp, Arabidopsis thaliana
caccgagctcactagttatcggagacgaattctg

SEQ ID NO: 52 - DNA, 31 bp, Arabidopsis thaliana
ctcggatccaaattagtcattcaacaaaaga

SEQ ID NO: 53 - DNA, 395 bp, Tobacco rattle virus
atgtcccgaagacattaaactacggttctttaagtagatccgtgtctgaagttttaggttcaatttaaacctacgagattgacattctcgactgatcttgattgatcggtaagtcttttgtaatttaattttctttttgattttattttaaattgttatctgtttctgtgtatagactgtttgagatcggcgtttggccgactcattgtcttaccataggggaacggactttgtttgtgttgttattttatttgtattttattaaaattctcaacgatctgaaaaagcctcgcggctaagagattgttggggggtgagtaagtacttttaaagtgatgatggttacaaaggcaaaaggggtaaaacccctcgcctacgtaagcgttattacgccc

SEQ ID NO: 54 - DNA, 799 bp, Arabidopsis thaliana
cgccctgagtgcttgcggcagcgtgaagcttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggccggt

SEQ ID NO: 55 - DNA, 263 bp, Arabidopsis thaliana
caaaagccatgcacaacaacacgtactcacaaaggcgtcaatcgagcagcccaaaacattcaccaactcaacccatcatgagcccacacatttgttgtttctaacccaacctcaaactcgtattctcttccgccacctcatttttgtttatttcaacacccgtcaaactgcatcccaccccgtggccaaatgttcatgcatgttaacaagacctatgactataaatatctgcaatctcggcccaagttttcatcatcaagaac